(19) United States
(12) Patent Application Publication
(10) Pub. No.: US 2024/0266060 A1
(43) Pub. Date: Aug. 8, 2024

(54) SYSTEMS AND METHODS FOR GENETICS-BASED ANALYTICS OF HEALTH, FITNESS AND SPORTS PERFORMANCE

(71) Applicant: Cipher Genetics, Inc., Bellevue, WA (US)

(72) Inventors: Gerrida UYS, Cape Town (ZA); Mariette Conning, Strand (ZA)

(21) Appl. No.: 18/409,116

(22) Filed: Jan. 10, 2024

Related U.S. Application Data
(63) Continuation of application No. PCT/US2022/037590, filed on Jul. 19, 2022.
(60) Provisional application No. 63/223,707, filed on Jul. 20, 2021.

(51) Int. Cl. G16H 50/30; A63B 24/00; G16B 20/20; G16H 10/40; G16H 15/00

(52) U.S. Cl. CPC G16H 50/30; A63B 24/0062; A63B 24/0075; G16B 20/20; G16H 10/40; G16H 15/00

(57) ABSTRACT

The present disclosure provides systems and methods for determining a performance or health risk state of a subject. A method for determining a performance or health risk state of a subject may comprise: (a) receiving genetic information of the subject obtained by assaying a biological sample obtained or derived from the subject; (b) receiving environmental information of the subject comprising contextual data, activities, or physiological measurements of the subject; (c) processing the genetic information and the environmental information to determine a performance or health risk state of the subject; and (d) outputting an electronic report indicative of the performance or health risk state of the subject.

### CROSS-REFERENCE

This application is a continuation of International Application No. PCT/US2022/037590, filed Jul. 19, 2022, which claims the benefit of U.S. Application No. 63/223,707, filed Jul. 20, 2021, each of which is incorporated by reference herein in its entirety.

### BACKGROUND

A specific health or performance status in a person may be determined by the combined effect of certain environmental conditions acting on a genetic background. It may be summarized by the equation Genetics (G)+Change in environment (Δ E)=Health (H).

### SUMMARY

Genetic data sets, for example, single nucleotide polymorphism (SNP) genotyping data, may be widely used to calculate polygenic risk scores for certain health conditions or performance traits. While polygenic risk scores give a good estimation of how a person's genetic risk (G) impacts a specific health condition or performance trait (H), it may not take the environmental contribution (Δ E) into consideration.

Commercial genetic reports may provide genotype results with risk or performance status coupled with insights based off academic literature on numerous health and fitness traits. These reports may be rich in information but may be difficult to understand and follow through, because the impact of these plans (the change in environment on health, fitness, and performance status) may be difficult to measure and show in an engaging way to a user. Genetics may play a significant role in a person's health and fitness journey (for example, a person's genetic make-up may influence their physiological performance in different sports by more than 50%). However, health and fitness decisions may be based without fully evaluating the genetics component, acting on it, or following through with it. Therefore, the health impact that genetics should bring may not be fully realized, with commercial genetic reports perceived to be of low value for what is an expensive test and technology.

Continuous physiological monitoring may enable real time insights to a range of physiological measures, for example, cardiorespiratory fitness, resting heart rate, stress levels, sleep quality, training load, blood pressure, and blood glucose levels. Wearable devices may offer an affordable solution that gives an array of trackable health and fitness performance scores. Similar to genetic recommendations, the insights based of these measures may be complicated to understand, translate, and incorporate into everyday health and fitness programs. Insights from wearable devices and applications may be retrospective, and may not provide predictive capabilities. Algorithms generating metrics and insights may be largely based off population averages, that inhibits personalization and accuracy.

To determine a more accurate health and performance status (H) at any given time, the environmental contribution (Δ E) to a person's genetic risk (G) may be determined through contextual information and continuous physiological monitoring. Continuously measuring the impact that lifestyle choices have on a person's genetic background and expressing that impact by means of a personal and easily interpretable health or performance-related score, may lead to improved adherence and adoption of genetic-based health and fitness recommendations.

Physiological monitoring and contextual information may serve as input to baseline genetic scores to estimate the environmental impact on gene expression that modifies the health or performance phenotype. A need therefore exists for an easy-to-use- and -understand technology that seamlessly integrates genetic information with physiological and contextual information, to enable real time insights and predictive metrics of a person's health and performance indicators, personalized for the individual.

The present disclosure provides methods and systems that integrate genetic data, contextual data, physiological biomarkers and/or continuous physiological data (e.g., which may be obtained from electronic devices such as wearable devices), to output easily interpretable health, fitness and sports performance-related scores through modifier algorithm calculations. The methods and systems of the present disclosure may be based on the concept of a modifier risk score for individual health conditions or performance traits, illustrated in FIG. 2. Each modifier score may comprise a static genetic contribution (expressed as a polygenic risk score, for example a pathway score) and a dynamic time-varying action contribution (measured through continuous physiological monitoring and contextual information). The action contribution may represents the impact that lifestyle choices have on the genetic background. The combined modifier score may be a real time, moving score that indicates a subject's level of risk. A risk manifests when the combined modifier score reaches a threshold. Modifier risk scores may enable daily recommendations to mitigate further risk, as well as predictive capabilities to indicate favorable or unfavorable outcomes given a certain health or training regime.

In an aspect, the present disclosure provides a computer-implemented method for determining a performance or health risk state of a subject, comprising: (a) receiving genetic information of the subject, wherein the genetic information is obtained by assaying a biological sample obtained or derived from the subject: (b) receiving environmental information of the subject, wherein the environmental information comprises contextual data, activities, or physiological measurements of the subject: (c) processing the genetic information and the environmental information to determine a performance or health risk state of the subject; and (d) outputting an electronic report indicative of the performance or health risk state of the subject.

In some embodiments, the performance or health risk state comprises a performance or health risk score, number, or quantitative metric of the subject. In some embodiments, the electronic report further comprises subject-specific health and fitness recommendations, such as how to improve a score or decrease a risk (e.g., generated based at least in part on the performance or health risk state or the performance or health risk score, number, or quantitative metric of the subject).

In some embodiments, the genetic information comprises nucleic acid sequence data. In some embodiments, the nucleic acid sequence data comprises deoxyribonucleic acid (DNA) sequence data, ribonucleic acid (RNA) sequence data, or a combination thereof. In some embodiments, the genetic information comprises genetic variants of the subject. In some embodiments, the genetic variants comprise at least one of: single nucleotide polymorphisms (SNPs), copy number variants (CNVs), insertions or deletions (indels), fusions, and translocations.

In some embodiments, the biological sample is selected from the group consisting of saliva, cheek swab, blood, plasma, serum, urine, and a combination thereof. In some embodiments, the assaying comprises at least one of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay. In some embodiments, the assaying comprises at least two of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay. In some embodiments, the activities comprise at least one of: exercising, playing a sport, walking, running, sitting, standing, lying down, and sleeping. In some embodiments, the physiological measurements comprise vital sign measurements of the subject. In some embodiments, the vital sign measurements comprise at least one of: heart rate, heart rate variability, systolic blood pressure, diastolic blood pressure, respiratory rate, blood oxygen concentration (SpO2), carbon dioxide concentration in respiratory gases, a hormone level, sweat analysis, blood glucose, body temperature, impedance, conductivity, capacitance, resistivity, electromyography, galvanic skin response, neurological signals, and immunology markers. In some embodiments, the physiological measurements comprise sports performance measurements. In some embodiments, the sports performance measurements comprise at least one of: VO2max, blood lactate, lactate threshold, training load, training stress scores, times spent in aerobic and anaerobic heart rate zones, pace, power, distance, and time. In some embodiments, the physiological measurements comprise a physiological metric that measures an effect of an external influence on a human body. In some embodiments, the activities or the physiological measurements are obtained using an electronic device (e.g., a wearable device).

In some embodiments. (c) further comprises determining the performance or health risk state of the subject based at least in part on one or more of: a diagnosis of a disease or disorder, a prognosis of a disease or disorder, a risk of having a disease or disorder, a risk of sports-related injury, a treatment history of a disease or disorder, a history of previous treatment for a disease or disorder, a history of prescribed medications, a history of prescribed medical devices, age, height, weight, sex, smoking status, injury risk, training load status, fitness level, race or match readiness, and one or more symptoms of the subject. In some embodiments, the one or more symptoms comprise chronic fatigue, weight loss, nausea, insomnia, or a combination thereof.

In some embodiments, the method further comprises generating a health regimen or a training regimen for the subject based at least in part on the performance or health risk state determined in (c). In some embodiments, the health regimen comprises recommendations to prevent onset of a disease or disorder, delay onset of a disease or disorder, reverse a disease or disorder, prevent injury, or maintain a physiological or health state of the subject. In some embodiments, the health regimen comprises recommendations related to one or more of: diet, exercise, sports training, supplements, functional tests, blood tests, brain management, behavior change, skin care, environmental exposure, stress management, and mental health. In some embodiments, the training regimen comprises a training program or a training rehabilitation program. In some embodiments, the electronic report is indicative of the health regimen. In some embodiments, the electronic report is presented on a graphical user interface of an electronic device of a user. In some embodiments, the user is the subject. In some embodiments, the electronic report is displayed through a user interface (e.g., providing a full view of an individual's context). In some embodiments, the user interface is configured to receive user input. In some embodiments, the user interface is presented via a software application (e.g., a mobile software application).

In some embodiments, the method further comprises transmitting the electronic report to a remote user. In some embodiments, the method further comprises transmitting an electronic health or fitness score and/or subject-specific health and fitness recommendations to a remote user. In some embodiments, the remote user is a clinical practitioner, a nutrigenetics counselor, a sports coach, a team manager, or an individual. In some embodiments, the method further comprises storing the electronic report on a remote server.

In some embodiments. (c) comprises processing the genetic information and the environmental information using a trained algorithm to determine the performance or health risk state of the subject. In some embodiments, the trained algorithm comprises a supervised machine learning algorithm. In some embodiments, the supervised machine learning algorithm comprises a deep learning algorithm, a support vector machine (SVM), a neural network, a Gaussian naïve Bayes model, a naïve Bayes model, or a Random Forest. In some embodiments, the trained algorithm comprises an unsupervised machine learning algorithm. In some embodiments, the unsupervised machine learning algorithm comprises a k-means clustering model or principal component analysis. In some embodiments, the trained algorithm is configured to determine the performance or health risk state of the subject at an accuracy of at least about 80%. In some embodiments, the trained algorithm is configured to determine the performance or health risk score of the subject at an accuracy of at least about 80%.

In some embodiments, the electronic report comprises a graphical representation of the performance or health risk state of the subject. In some embodiments, the graphical representation comprises a time-series graph illustrating performance or health risk scores of a subject over time (e.g., daily). In some embodiments, the method further comprises using the electronic report to provide the subject with a therapeutic intervention. In some embodiments, the therapeutic intervention comprises a drug. In some embodiments, the performance or health risk state of the subject comprises a risk score. In some embodiments, the method further comprises using the risk score to modify a physiological estimation or measurement of the subject, and determining the performance or health risk state based at least in part on the modified physiological estimation or measurement of the subject. In some embodiments, the method further comprises generating a health regimen or a training regimen for the subject to decrease risk or improve performance, based at least in part on the modified physiological estimation or measurement of the subject. In some embodiments, the health regimen comprises recommendations to prevent onset of a disease or disorder, delay onset of a disease or disorder, reverse a disease or disorder, prevent injury, or maintain a physiological or health state of the subject. In some embodiments, the health regimen comprises recommendations related to one or more of: diet, exercise, sports training, supplements, functional tests, blood tests, brain management, behavior change, skin care, environmental exposure, stress management, and mental health. In some embodiments, the training regimen comprises a training program or a training rehabilitation program. In some embodiments, the method further comprises generating an updated performance or health risk state responsive to the subject following the health regimen. In some embodiments, at least (b) and (c) are continuously performed in real-time.

In another aspect, the present disclosure provides a system for determining a performance or health risk state of a subject, comprising: a database configured to store genetic information of the subject, wherein the genetic information is obtained by assaying a biological sample obtained or derived from the subject, and environmental information of the subject, wherein the environmental information comprises contextual data, activities, or physiological measurements of the subject; and one or more computer processors operatively coupled to the database, wherein the one or more computer processors are individually or collectively programmed to: (i) process the genetic information and the environmental information to determine a performance or health risk state of the subject; and (ii) electronically output a report indicative of the performance or health risk state of the subject.

In another aspect, the present disclosure provides a non-transitory computer-readable medium comprising machine-executable code that, upon execution by one or more computer processors, implements a method for determining a performance or health risk state of a subject, the method comprising: (a) receiving genetic information of the subject, wherein the genetic information is obtained by assaying a biological sample obtained or derived from the subject: (b) receiving environmental information of the subject, wherein the environmental information comprises contextual data, activities, or physiological measurements of the subject: (c) processing the genetic information and the environmental information to determine a performance or health risk state of the subject; and (d) outputting an electronic report indicative of the performance or health risk state of the subject.

Another aspect of the present disclosure provides a non-transitory computer-readable medium comprising machine-executable code that, upon execution by one or more computer processors, implements any of the methods above or elsewhere herein.

Another aspect of the present disclosure provides a system comprising one or more computer processors and computer memory coupled thereto. The computer memory comprises machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a method 100 for determining a performance or health risk state of a subject.

FIG. 2 shows an example of a concept of modifier risk scores, including a threshold (purple line) for manifestation of a risk (or a goal, depending on the application), and purple arrows showing where a subject is in terms of their risk threshold, presented as a modified risk score.

FIG. 3 shows an example of components and data flow of modifier algorithms.

FIG. 4 shows an example of a system diagram for the modifier algorithm solution.

FIGS. 5A-5C show examples of a software application user interface for login and sign up flow.

FIGS. 6A-6C show examples of a software application user interface for contextual information capture flow.

FIGS. 7A-7E show examples of a software application user interface for genetic and wearable data linking flow.

FIGS. 8A-8E show examples of a software application user interface, including a dashboard displaying summary overview of fitness metrics, calculated by modifier algorithms, together with drill down screens of Today's Training, Recovery time, capturing of contextual recovery activities, and Fitness progress.

FIGS. 9A-9D show examples of a software application user interface, including an injury risk feature page, showing daily genetically adapted risk for individual injuries (ACL, Achilles tendon, stress fracture, rotator cuff) and genetically adapted injury risk with physiological fitness over time.

FIG. 10 shows an example of individual single nucleotide polymorphisms (SNPs) used to calculate genetic pathway risk scores for four common overuse injury types, together with heritability scores (indicated in percentages) and an overall injury risk pathway.

FIG. 11 shows an example of a probability density function of calculated pathway scores.

FIG. 12 shows an example of a cumulative distribution function of the corresponding pathway scores shown in FIG. 11.

FIGS. 13A-13D show examples of shifts in injury risk depending on pathway score, including a first injury risk distribution determined without genetics (red) and a second injury risk distribution determined with genetics (green).

FIG. 14 shows a computer system that is programmed or otherwise configured to implement methods provided herein.

FIG. 15 shows an example of a framework for modelling injuries using genetic modifiers.

FIG. 16 shows an example of a modified TQR algorithm.

FIG. 17 shows an example of sensitivity of an injury risk model and a standard ACWR model.

FIG. 18 shows an example of specificity of an injury risk model and a standard ACWR model.

FIG. 19 shows an example of a time series of an athlete's injury risk as determined by an injury risk model and a standard ACWR model.

FIGS. 20A-20G show examples of various views of a software application user interface from a user perspective of a player (e.g., subject).

FIGS. 21A-21G show examples of various dashboard views of a software application user interface from a user perspective of a player (e.g., subject).

FIGS. 22A-22L show examples of various views of a software application user interface from a user perspective of a manager.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a,” “an,” and “the” include plural references, unless the context clearly dictates otherwise. For example, the term “a biological sample” includes a plurality of biological samples, including mixtures thereof.

As used herein, the term “subject.” generally refers to an organism having testable or detectable genetic, nutrigenetic, or other health or other physiological parameter or information. A subject may be a person. The subject may be a vertebrate, such as, for example, a mammal. Non-limiting examples of mammals include humans, simians, farm animals, sport animals, and pets. The subject may be an organism, such as an animal, a plant, a fungus, an archaea, or a bacteria. The subject may be a human. The subject may be a non-human. The subject may have or be suspected of having a heath or physiological condition, such as a disease. In some examples, the subject is a patient. As an alternative, the subject may be asymptomatic with respect to the health or physiological condition (e.g., disease).

The term “biological sample.” as used herein, generally refers to a biological sample that may be obtained from a subject. Samples obtained from subjects may comprise a biological sample from a human, animal, plant, fungus, or bacteria. The sample may be obtained from a subject with a disease or disorder, from a subject that is suspected of having the disease or disorder, or from a subject that does not have or is not suspected of having the disease or disorder. The disease or disorder may be an infectious disease, an immune disorder or disease, a cancer, a genetic disease, a degenerative disease, a lifestyle disease, an injury, a rare disease, or an age related disease. The infectious disease may be caused by bacteria, viruses, fungi, and/or parasites. The sample may be taken before and/or after treatment of a subject with a disease or disorder. Samples may be taken during a treatment or a treatment regime. Multiple samples may be taken from a subject to monitor the effects of the treatment over time. The sample may be taken from a subject having or suspected of having a disease or disorder for which a definitive positive or negative diagnosis is not available via clinical tests.

A sample may be obtained from a subject suspected of having a disease or a disorder. The subject may be experiencing unexplained symptoms, such as fatigue, nausea, weight loss, aches and pains, weakness, or memory loss. The subject may have explained symptoms. The subject may be at risk of developing a disease or disorder due to factors such as familial history, age, environmental exposure, lifestyle risk factors, or presence of other known risk factors.

A sample may comprise a biological sample from a subject (e.g., human subject), such as saliva, cheek swab, blood, plasma, serum, cells, tissue (e.g., normal or tumor), urine, stool (feces), or derivatives or combinations thereof. The sample may be a tissue sample, such as a tumor sample. The sample may be a cell-free sample, such as a blood (e.g., whole blood), sweat, saliva or urine sample. The biological sample may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, potassium dichromate, or EDTA).

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include deoxyribonucleic acid (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

The nucleic acid molecules may comprise deoxyribonucleic acid (DNA), ribonucleic acid (RNA) molecules, or a combination thereof. The DNA or RNA molecules may be extracted from the sample by a variety of methods, such as a FastDNA Kit protocol from MP Biomedicals. The extraction method may extract all DNA molecules from a sample. Alternatively, the extraction method may selectively extract a portion of DNA molecules from a sample, e.g., by targeting certain genes in the DNA molecules. Alternatively, extracted RNA molecules from a sample may be converted to DNA molecules by reverse transcription (RT). In some embodiments, after obtaining the sample, the sample may be processed to generate a plurality of genomic sequences. For example, processing the sample may comprise extracting a plurality of nucleic acid (DNA or RNA) molecules from the sample, and sequencing the plurality of nucleic acid (DNA or RNA) molecules to generate a plurality of nucleic acid (DNA or RNA) sequence reads.

The sequencing may be performed by any suitable sequencing method, such as massively parallel sequencing (MPS), paired-end sequencing, high-throughput sequencing, next-generation sequencing (NGS), shotgun sequencing, single-molecule sequencing (e.g., Pacific Biosciences of California), nanopore sequencing (e.g., Oxford Nanopore), semiconductor sequencing, pyrosequencing (e.g., 454 sequencing), sequencing-by-synthesis (SBS), sequencing-by-ligation, and sequencing-by-hybridization, or RNA-Seq (Illumina). Sequence identification may be performed using a genotyping approach such as an array. As an example, an array may be a microarray (e.g., Affymetrix or Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing or genotyping. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies. Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing or genotyping. The PCR may comprise targeted amplification of one or more genomic loci, such as genomic loci corresponding to one or more diseases or disorders such as cancer markers (e.g., BRCA 1 and 2). The sequencing or genotyping may comprise use of simultaneous reverse transcription (RT) and polymerase chain reaction (PCR), such as a OneStep RT-PCR kit protocol provided by Qiagen. NEB. Thermo Fisher Scientific, or Bio-Rad.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase. For example, sequencing or genotyping of DNA molecules may be performed with or without amplification of DNA molecules.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA or RNA molecule may be traced back to the sample (and the environment or the subject) from which the DNA or RNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the plurality of genomic sequences. For example, the sequence reads may be filtered for quality, trimmed to remove low quality, or aligned to one or more reference genomes (e.g., a human genome).

In some embodiments, after obtaining the biological sample, the biological sample may be processed to generate a proteome, metabolome, or any combination thereof. For example, processing the biological sample may comprise extracting a plurality of proteins from the biological sample, and analyzing the plurality of proteins to identify and/or quantify the plurality of proteins, thereby generating a proteome of the biological sample. As another example, processing the biological sample may comprise extracting a plurality of metabolites from the biological sample, and analyzing the plurality of metabolites to identify and/or quantify the plurality of metabolites, thereby generating a metabolome of the biological sample. The extraction method may extract all proteins and/or metabolites from a biological sample. Alternatively, the extraction method may selectively extract a portion of proteins and/or metabolites from a biological sample, e.g., by use of binding reagents such as probes or antibodies to target certain proteins and/or metabolites.

As used herein, a user can be an end-consumer, a company having at least one product that can analyze human genetic data to generate health-related recommendations and other information to an end-consumer; an entity that does not have any product but may also utilize the human genetic data for other purposes such as research; a subject from which the biological samples and/or nutrigenetic data are obtained: or a physician, nurse, nutrigenetic counselor, or other clinical provider. Genetic data may include nutrigenetic data which may comprise nutrigenetic aberrations. Genetic data may include sports performance, energy metabolism, and sports nutrition data.

The terms “nutrigenetic” and “nutrigenomic,” as used herein, generally refer to nutritional genetic or nutritional genomic information, such as relationships between a genome, nutrition, and health of a subject. For example, nutrigenetic analysis may be related to identifying or predicting heterogeneous or differential response of a subject to diet and nutrients based on analysis of nucleic acid sequences having gene variants, while nutrigenomics analysis may be related to the influence of diet and nutrients on the gene expression of a subject.

The term “nutrigenetic aberration,” as used herein, generally refers to a nutrition-related aberration in a genome of a subject, such as, for example, a nutrigenetic variant. A nutrigenetic aberration may be, for example, a genetic variation that has a relationship (e.g., a causative relationship or a highly correlated relationship, such as, for example, a correlation at an R²>0.8 or 0.9) with a nutrition and health of a subject, such as a single nucleotide polymorphisms (SNP), copy number variation (CNV), insertion or deletion (indel), fusion, or translocation. A nutrigenetic variant may be, for example, a genetic variant associated with differential response to nutrients (e.g., differential gene expression or DNA methylation).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

A specific health or performance status in a person may be determined by the combined effect of certain environmental conditions acting on a genetic background. It may be summarized by the equation Genetics (G)+Change in environment (Δ E)=Health (H).

Genetic data sets, for example, single nucleotide polymorphism (SNP) genotyping data, may be widely used to calculate polygenic risk scores for certain health conditions or performance traits. While polygenic risk scores give a good estimation of how a person's genetic risk (G) impacts a specific health condition or performance trait (H), it may not take the environmental contribution (Δ E) into consideration.

Commercial genetic reports may provide genotype results with risk or performance status coupled with insights based off academic literature on numerous health and fitness traits. These reports may be rich in information but may be difficult to understand and follow through, because the impact of these plans (the change in health, fitness, and performance status due to changes in environment) may be difficult to measure and show in an engaging way to a user. Genetics may play a significant role in a person's health and fitness journey (for example, a person's genetic make-up may influence their physiological performance in different sports by more than 50%). However, health and fitness decisions may be based without fully evaluating the genetics component, acting on it, or following through with it. Therefore, the health impact that genetics should bring may not be fully realized, with commercial genetic reports perceived to be of low value for what is an expensive test and technology.

Continuous physiological monitoring may enable real time insights to a range of physiological measures, for example, cardiorespiratory fitness, resting heart rate, stress levels, sleep quality, training load, blood pressure, and blood glucose levels. Wearable devices may offer an affordable solution that gives an array of trackable health and fitness performance scores. Similar to genetic recommendations, the insights based of these measures may be complicated to understand, translate, and incorporate into everyday health and fitness programs. Insights from wearable devices and applications may be retrospective, and may not provide predictive capabilities. Algorithms generating metrics and insights may be largely based off population averages, that inhibits personalization and accuracy.

To determine a more accurate health and performance status (H) at any given time, the environmental contribution (A E) to a person's genetic risk (G) may be determined through contextual information and continuous physiological monitoring. Continuously measuring the impact that lifestyle choices have on a person's genetic background and expressing that impact by means of a personal and easily interpretable health or performance-related score, may lead to improved adherence and adoption of genetic-based health and fitness recommendations.

Physiological monitoring and contextual information may serve as input to baseline genetic scores to estimate the environmental impact on gene expression that modifies the health or performance phenotype. A need therefore exists for an easy-to-use- and -understand technology that seamlessly integrates genetic information with physiological and contextual information, to enable real time insights and predictive metrics of a person's health and performance indicators, personalized for the individual.

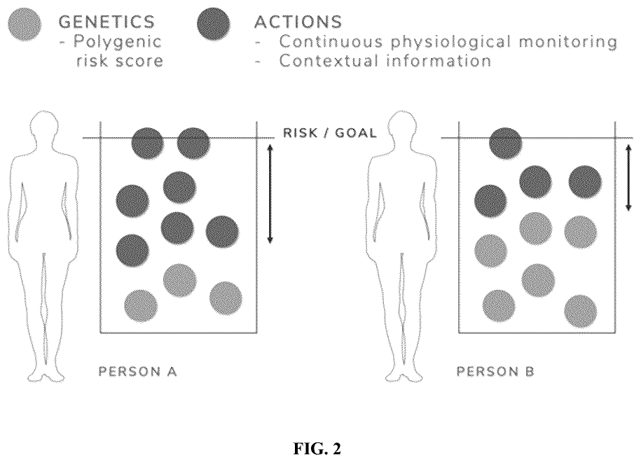

The present disclosure provides methods and systems that integrate genetic data, contextual data, physiological biomarkers and/or continuous physiological data obtained from wearable devices, to output easily interpretable health, fitness and sports performance-related scores through modifier algorithm calculations. The methods and systems of the present disclosure may be based on the concept of a modifier risk score for individual health conditions or performance traits, illustrated in FIG. 2. Each modifier score may comprise a static genetic contribution (expressed as a polygenic risk score, for example a pathway score) and a dynamic time-varying action contribution (measured through continuous physiological monitoring and contextual information). The action contribution may represents the impact that lifestyle choices have on the genetic background. The combined modifier score may be a real time, moving score that indicates a subject's level of risk. A risk manifests when the combined modifier score reaches a threshold. Modifier risk scores may enable daily recommendations to mitigate further risk, as well as predictive capabilities to indicate favorable or unfavorable outcomes given a certain health or training regime.

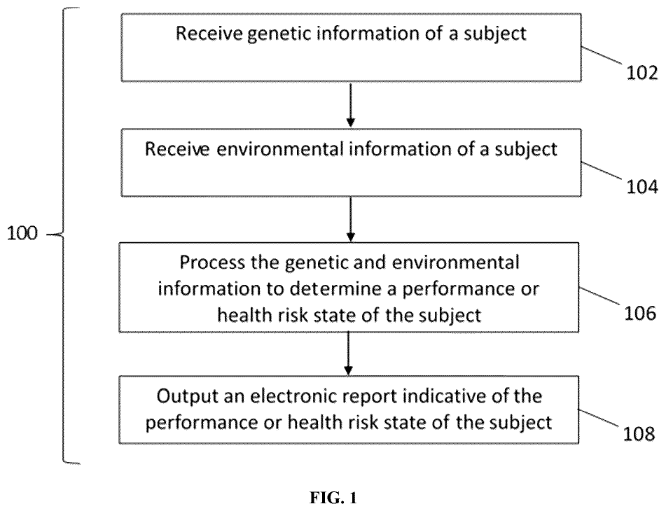

FIG. 1 shows an example of a method 100 for determining a performance or health risk state of a subject. The method 100 may comprise receiving genetic information of a subject (as in operation 102). The genetic information may be obtained by assaying a biological sample obtained or derived from the subject. The method 100 may comprise receiving environmental information of the subject (as in operation 104). The environmental information may comprise contextual data, activities, biochemical, or physiological measurements of the subject. The method 100 may comprise processing the genetic information and the environmental information to determine a performance or health risk state of the subject (as in operation 106). The performance or health risk state may comprise a performance or health risk score, number, or quantitative metric of the subject. The method 100 may comprise outputting an electronic report indicative of the performance or health risk state of the subject (as in operation 108). The electronic report may further comprise subject-specific health and fitness recommendations, such as how to improve a score or decrease a risk (e.g., generated based at least in part on the performance or health risk state or the performance or health risk score, number, or quantitative metric of the subject). The electronic report may comprise a graphical representation of the performance or health risk state of the subject, such as a time-series graph illustrating performance or health risk scores of a subject over time (e.g., daily). The electronic report may be displayed through a user interface (e.g., providing a full view of an individual's context). The user interface may be configured to receive user input. The user interface may be presented via a software application (e.g., a mobile software application).

FIG. 2 shows an example of a concept of modifier risk scores, including a threshold (purple line) for manifestation of a risk (or a goal, depending on the application), and purple arrows showing where a subject is in terms of their risk threshold, presented as a modified risk score.

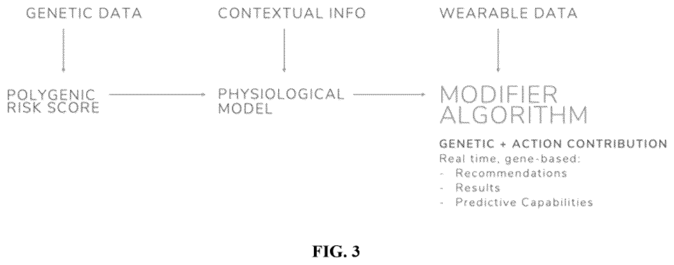

Modifier algorithms, used to calculate real time, moving modifier scores, may comprise the following data flow structure, as illustrated in FIG. 3, which shows an example of components and data flow of modifier algorithms.

Genetic data, for example, SNP genotyping results, may be used to calculate polygenic risk scores or pathway scores for multiple biochemical pathways. These scores may serve as input for various and diverse physiological models. Contextual input, for example age, sex, height, weight and previous injuries obtained, may serve as additional input for physiological models. Examples of physiological models include models of sport performance, such as VO2 max, lactate threshold, training load, and acute:chronic workload ratio (ACWR). Continuous physiological data, obtained from wearable devices, for example heart rate-derived data, may serve as real time input to the modifier algorithm and enables the calculation of daily modifier scores, that is a combined score showing the genetic contribution together with the environmental (or action) contribution. Additional physiological and biochemical markers, such as blood results (e.g., blood glucose results) and biomarkers, may also serve as an input for modifier algorithms.

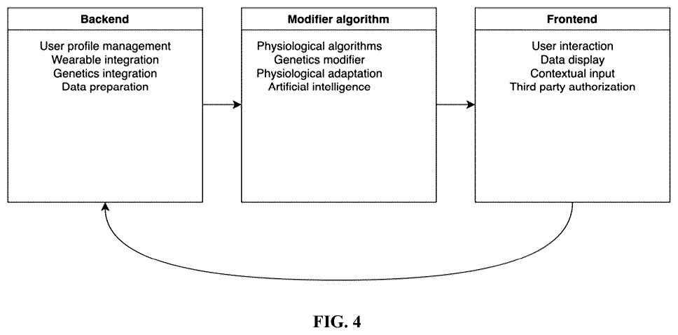

The present disclosure provides physiologically driven algorithms, artificial intelligence algorithms, a system architecture capturing wearable data, and a software (e.g., mobile, desktop, or tablet) application solution. All these modules may be integrated into a system giving the users their actionable insights. FIG. 4 shows an example of a system diagram for the modifier algorithm solution.

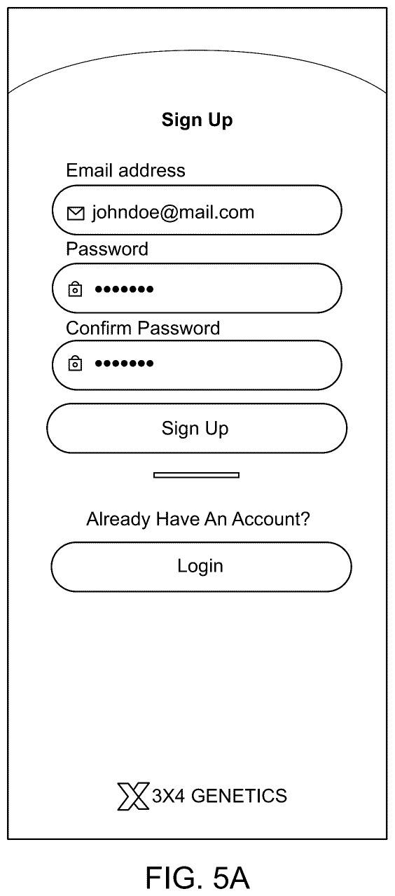

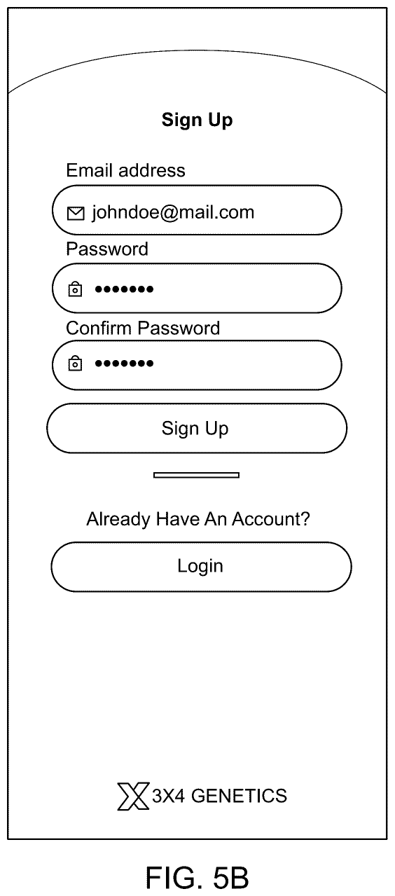

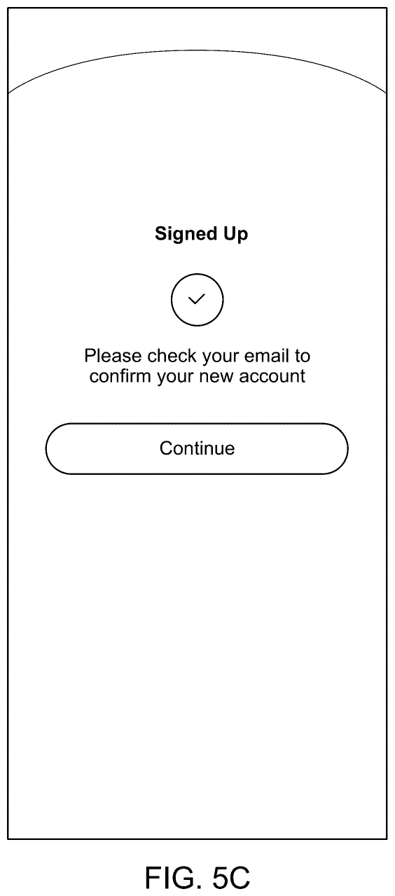

The mobile application solution (referred to as the frontend in above system diagram) may be used to communicate easily interpretable health and sports performance-related scores and actionable insights to the user, and follows a standard login and sign up process to create a user profile, as shown in FIGS. 5A-5C, which show examples of a software application user interface for login and sign up flow.

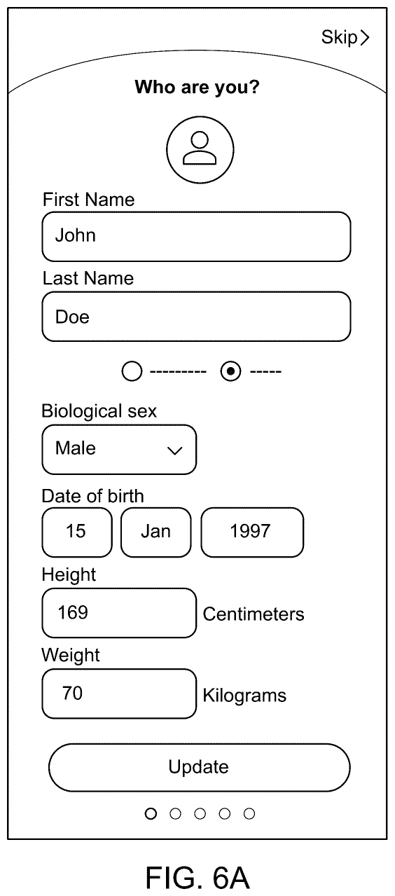

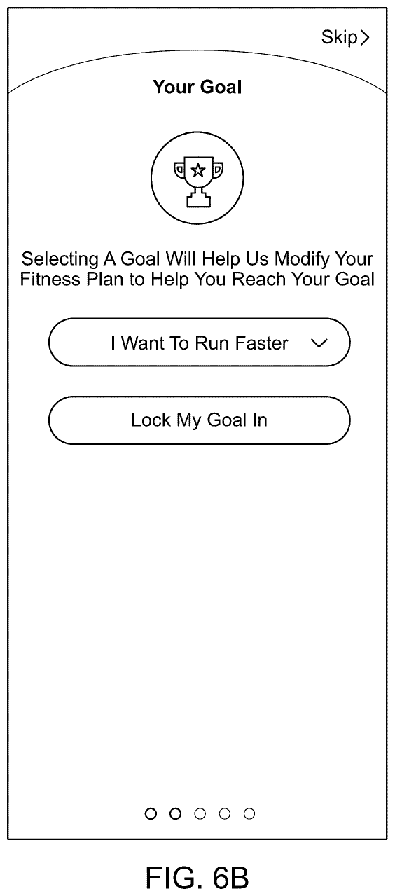

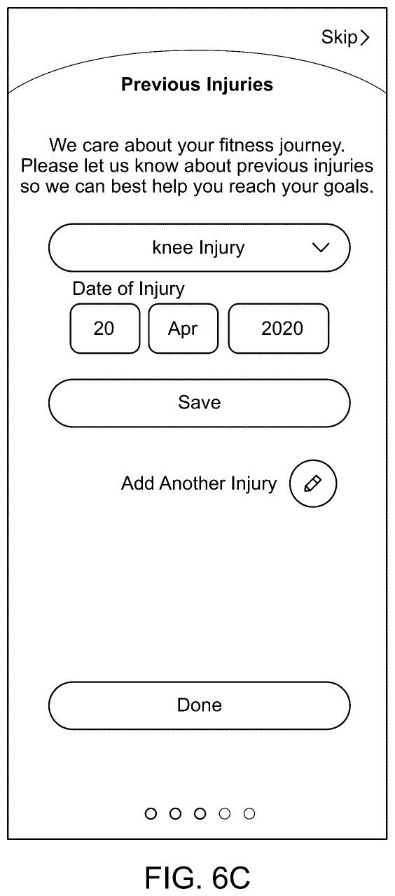

Contextual information, for example biometric information such as sex, age, weight, height, previous injury history and specific goals may be captured through the following screens shown in FIGS. 6A-6C, which show examples of a software application user interface for contextual information capture flow.

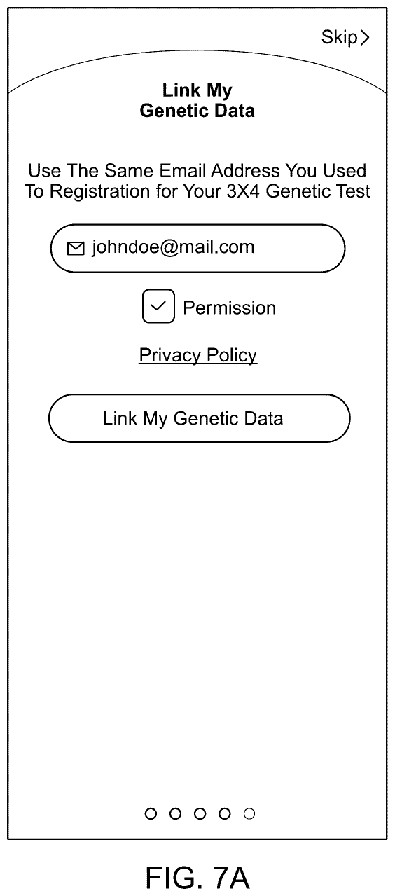

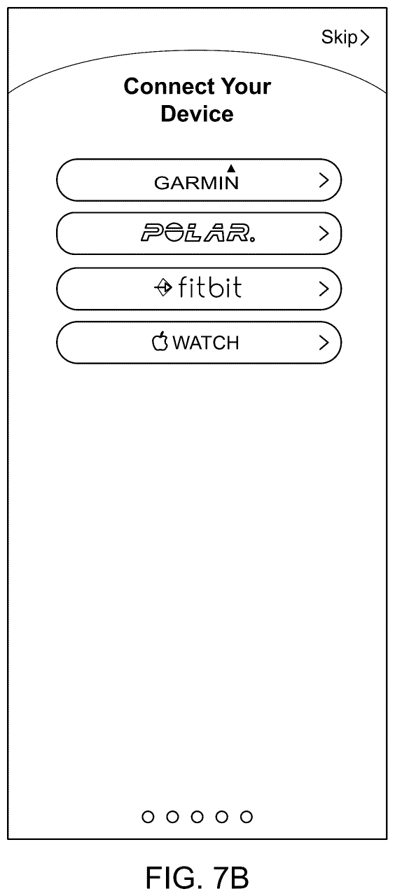

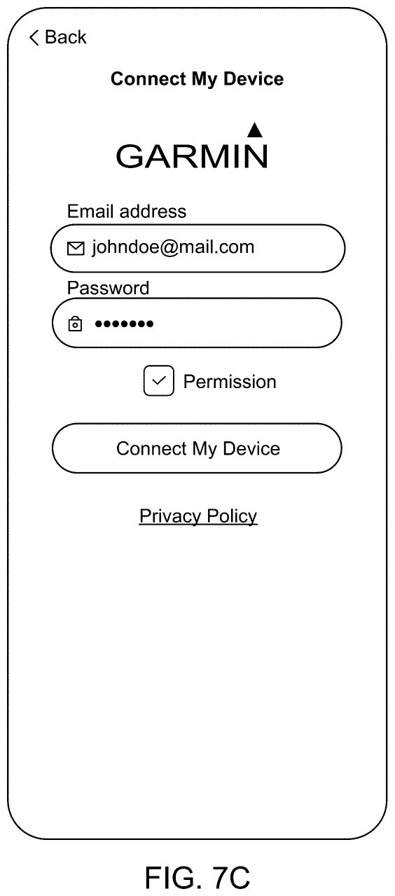

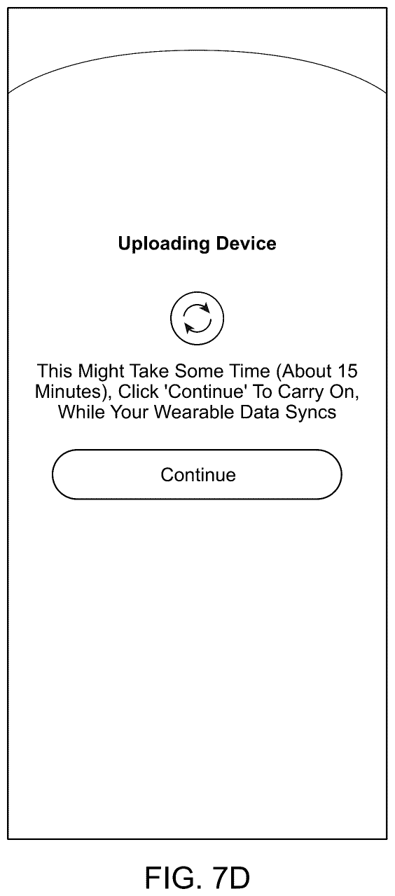

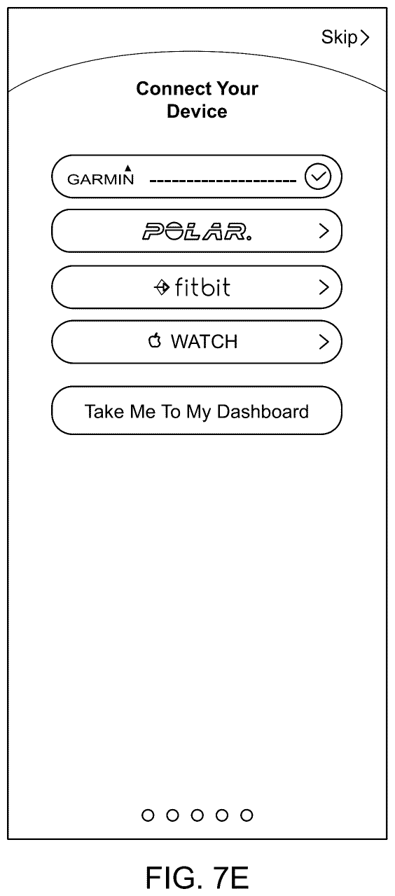

FIGS. 7A-7E show examples of a software application user interface for genetic and wearable data linking flow: A user's genetic data (SNP genotyping results in .txt format) may be linked to their profile on the backend. Genotyping results may serve as input for an algorithm that calculates polygenic risk scores or pathway scores for an individual for a range of biochemical pathways, including, but not limited to, the following: endurance, VO2 max trainability, slow twitch fibers, power, fast twitch fibers, anaerobic threshold, strength, recovery, inflammation, oxidative stress, muscle damage, injury, rotator cuff injury, anterior cruciate ligament (ACL) injury. IT band injury, stress fractures, knee osteoarthritis, and Achilles tendon injury.

A user's wearable device and data may be linked to their profile through third party authorization. Wearable data files containing heart rate, speed, power, duration, time, exercise type, elevation, lap details and other biometric data may be uploaded and serve as input for a range of physiological models and modifier algorithms on the backend.

Modifier scores for health, fitness and sports performance may be continuously updated through modifier algorithms as new wearable and contextual data are uploaded to the system. By displaying these score outputs as easily interpretable and understandable visual metrics on a mobile application user interface, a user may keep track of the impact that their lifestyle choices have on their genetic background, and how it impacts their health and performance.

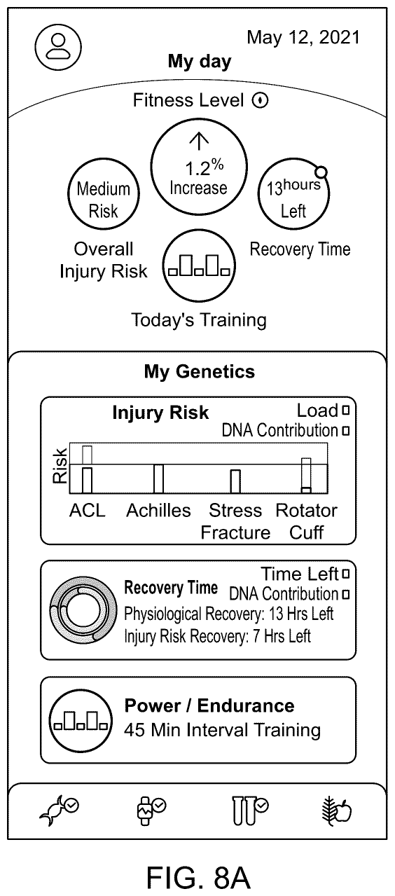

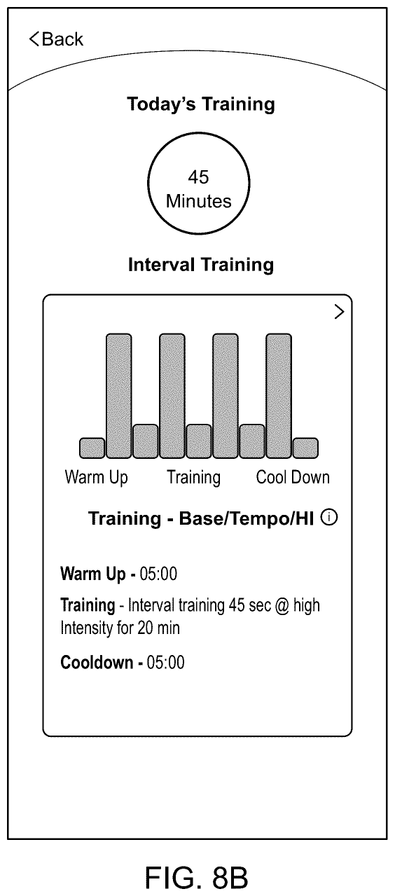

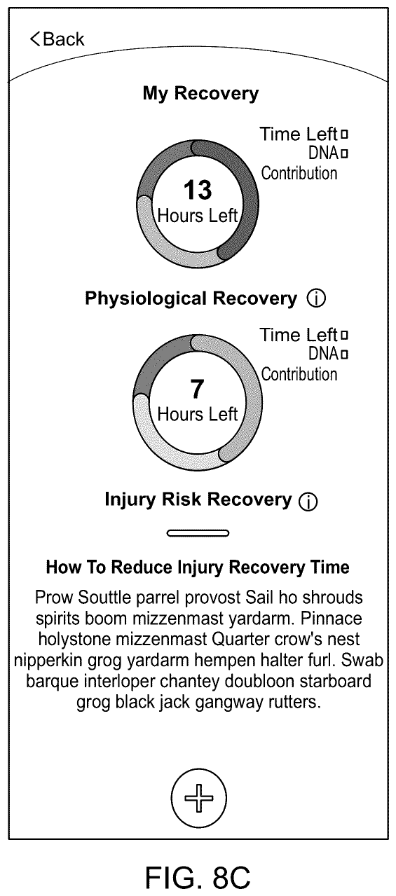

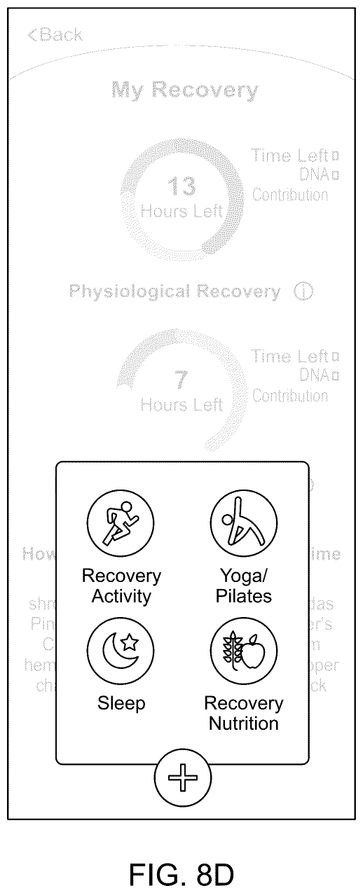

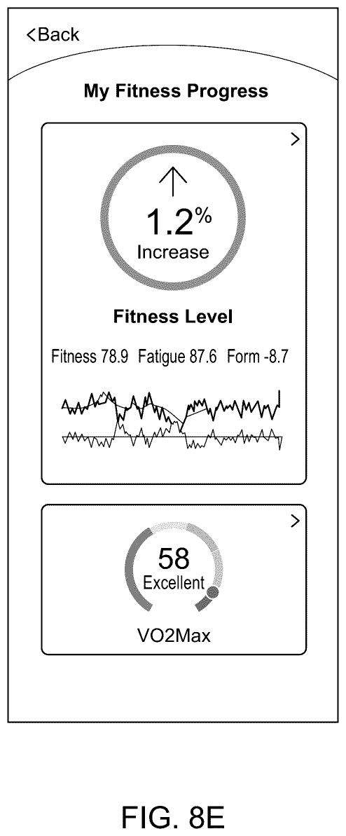

FIGS. 8A-8E show examples of a software application user interface, including a dashboard displaying summary overview of fitness metrics, calculated by modifier algorithms, together with drill down screens of Today's Training. Recovery time, capturing of contextual recovery activities, and Fitness progress. Dynamic metrics and recommendations may enable a more accurate and personal health and fitness journey compared to using only genetic risk scores and information, which may be offered by genetic testing companies through health reports, or physiological models based on population averages alone, which may be offered through wearable device applications and platforms.

The dashboard provides a summary overview of important fitness metrics, calculated through modifier algorithms. These scores and metrics may drive machine learning algorithmic recommendations and dynamic workout plans. An example is Today's Training, which gives a workout program tailored according to a person's genetic predisposition for certain exercise types, their training history, and physiological model outputs such as recovery time and injury risk, all captured by a combination of genetic, contextual and wearable data that drives a machine learning algorithm.

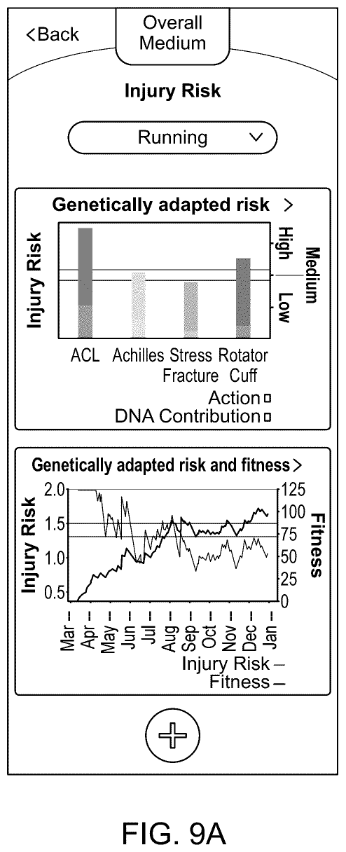

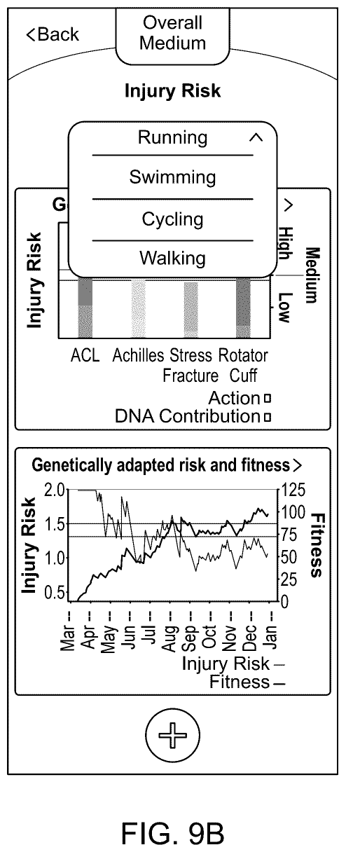

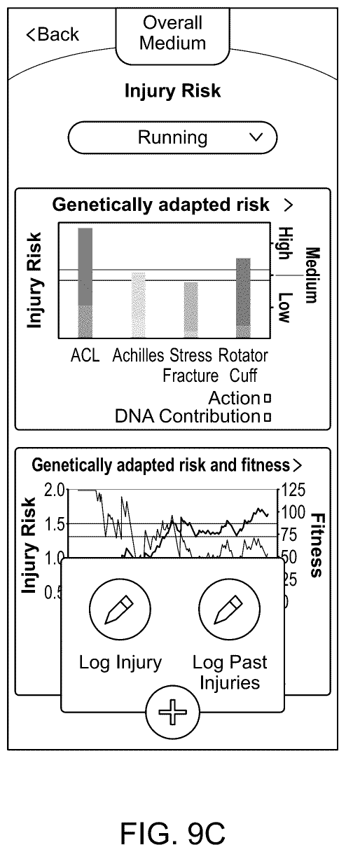

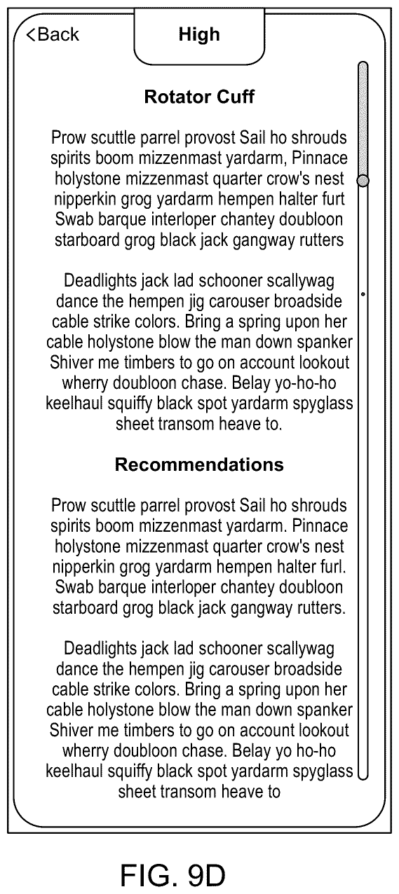

The injury risk feature may use SNP genotyping data to calculate biochemical pathway risk scores, related to common overuse sports injuries, for example rotator cuff injury, anterior cruciate ligament (ACL) injury, stress fractures. Achilles tendon injury, muscle damage, connective tissue injury, and a general injury pathway risk score. Individual pathway risk scores, together with contextual information such as previous injuries sustained (injury type and date of injury) and exercise type, plus heart rate-derived data, obtained from wearable data, may be transformed by modifier algorithms. These transformations may give the user a daily genetically adapted injury risk status based on genetic and action contribution, as well as exercise type selected, as shown in FIGS. 9A-9D, which show examples of a software application user interface, including an injury risk feature page, showing daily genetically adapted risk for individual injuries (ACL, Achilles tendon, stress fracture, rotator cuff) and genetically adapted injury risk with physiological fitness over time. Injury risk will differ depending on which exercise type is selected. Injuries may be manually logged, to adjust injury risk profiles. Recommendations to mitigate different injury risks are shown by clicking on a risk bar for that specific injury type.

Genetically adapted injury risk shows the user when they are reaching their risk threshold for a specific injury type, depending on the combination of their genetics, actions, and exercise type. The injury risk feature also shows genetic adapted injury risk and physiological fitness over time. The modifier algorithm illustrates how injury risk decrease over time as physiological adaptation occurs through the correct training stimuli over time.

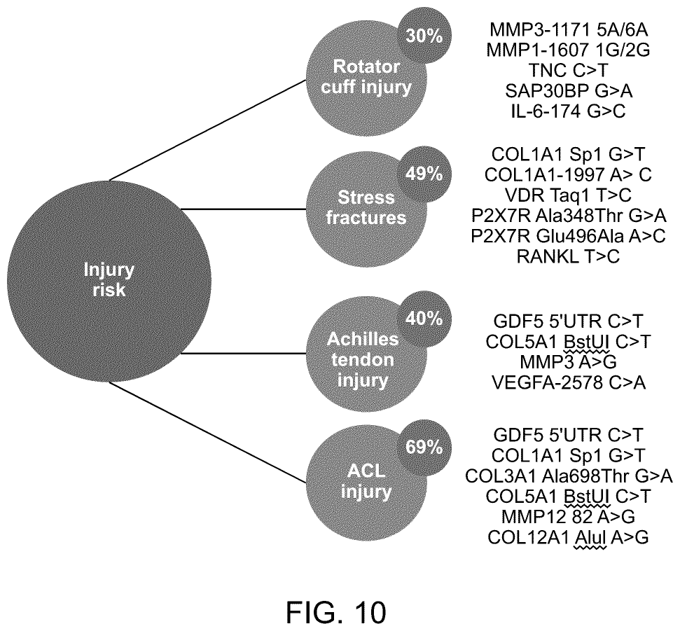

FIG. 10 shows an example of individual single nucleotide polymorphisms (SNPs) used to calculate genetic pathway risk scores for four common overuse injury types (rotator cuff injury, stress fractures. Achilles tendon injury and ACL injury), together with heritability scores (indicated in percentages) and an overall injury risk pathway.

The modifier algorithms for each injury type depends on the population distribution of the pathway risk scores—the factor by which a physiological measure is adjusted is determined by a cumulative distribution function (cdf) and a user's pathway risk score. Using a cdf may ensure that a pathway risk score is not given a higher/lower weight of influence given a specific distribution of pathway risk scores.

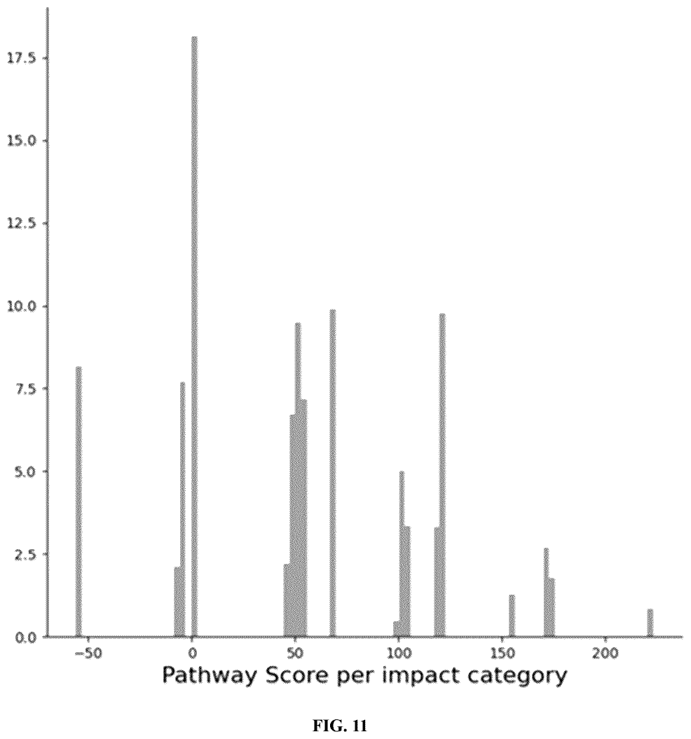

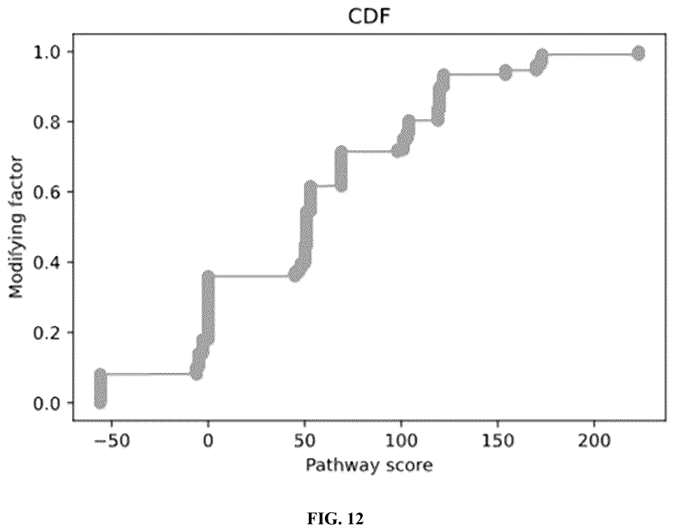

FIG. 11 shows an example of a probability density function of calculated pathway scores. FIG. 12 shows an example of a cumulative distribution function of the corresponding pathway scores shown in FIG. 11.

To calculate the injury risk of a specific pathway, the following process may be followed from raw data to a risk measure being shown on the mobile app. The first calculation may be performed to estimate the effect of a training session on an individual's physiology using metrics used for the general case. This is done by calculating the load of a session using the duration of the session and the intensity of the session. From the daily load, an acute load and chronic load measure is determined. Acute load is calculated over a short period of time, such as 7 days. Chronic load may be calculated using a longer period, which may vary according to different sports (e.g., 42 days). The ratio of Acute to Chronic load (ACWR) may be an indication of how much training is being done in relation to how much an individual can handle. If this ratio becomes too high, the risk of injury may increase.

The modifier algorithm may incorporate genetics to the equation, and this may be done by adapting both the session load and the ACWR. By how much these values are adapted is determined by an individual's pathway score, the heritability of a particular injury and the distribution of all possible pathway scores in a given population. The modifier algorithm may calculate a modifier score that is used to shift a physiological score up or down, depending on the three factors mentioned previously. This is illustrated in the figures below for 4 different types of injury. The risk of a person shifts either up or down in relation to the standard measure where genetics are not considered.

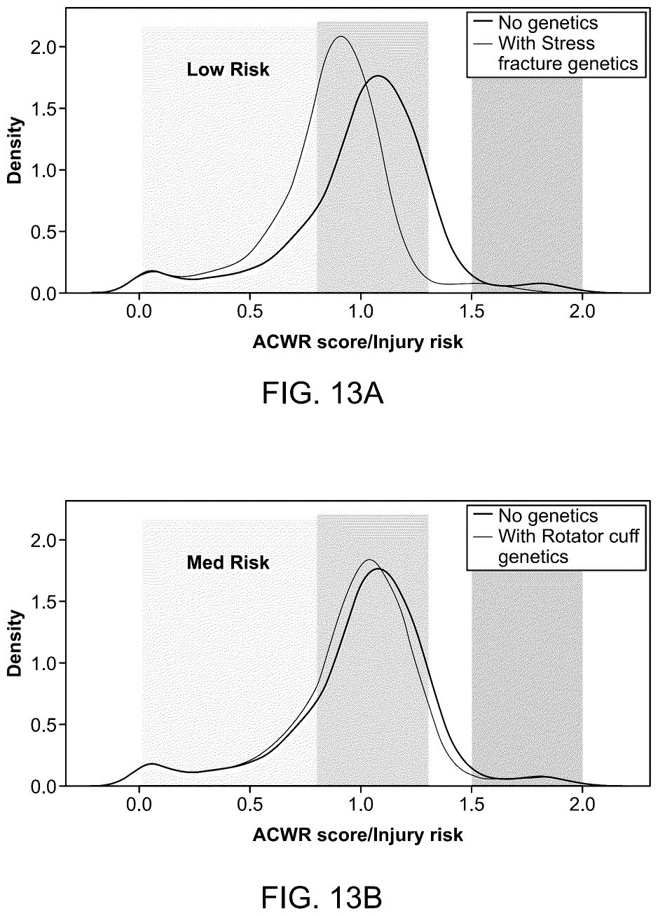

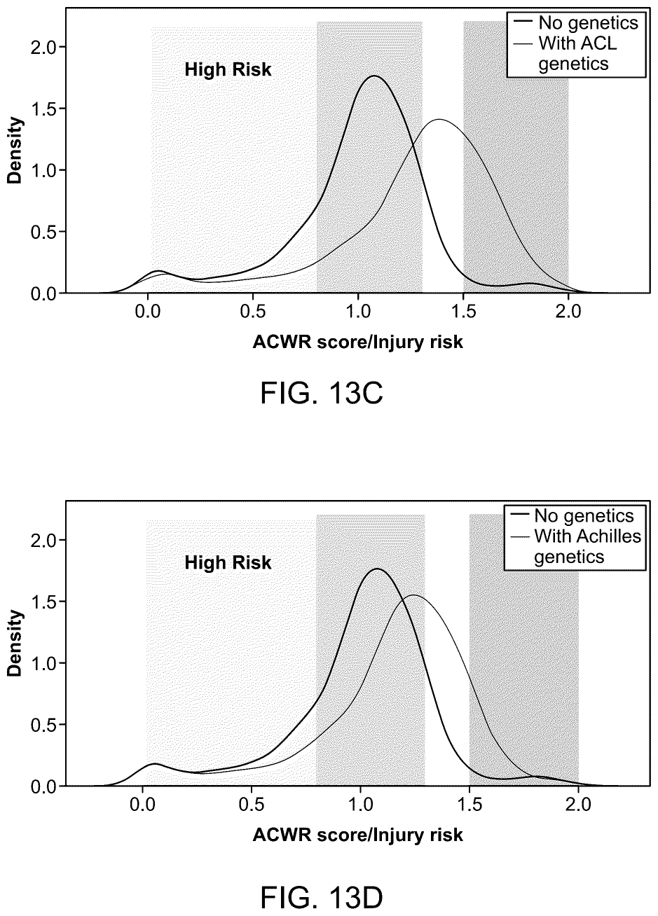

FIGS. 13A-13D show examples of shifts in injury risk depending on pathway score, including a first injury risk distribution determined without genetics (red) and a second injury risk distribution determined with genetics (green).

### User Portals and Platforms

Systems of the present disclosure may generate a profile of the subject, facilitate data exchange of the profile among end users (e.g., using a network such as a cloud network), store the profile in a database (e.g., a cloud network), and/or display an electronic report comprising the profile to an end user.

The system may facilitate data exchange of the profile among end users (e.g., using a network such as a cloud network) and/or store the profile in a database (e.g., a cloud network). The system may comprise a network interface that is in network communication with digital computers of different users. The network interface may include a portal or a platform, such as a user portal (e.g., for an end user to view profiles) or a clinician portal (e.g., for a clinician to view or annotate profiles). In some embodiments, a cloud-based method or system can be provided to a user for facilitating data exchange. The user can use a web-application to log in and access his data over a cloud-based computer system in the application, wherein the data is generated from processing at least one biological sample of the user. The data exchange and/or data storage may take into account privacy laws and policies, such as Health Insurance Portability and Accountability Act of 1996 (HIPAA) compliance and safeguarding of protected health information (PHI).

The systems and methods provided herein can include a user portal and/or a user platform that is configured to perform health, fitness, and sports performance analysis, display profiles and reports to a user and/or control access to profiles, reports, and/or data. The user portal and/or user platform may include a server that includes a digital processing device or a processor that can execute machine code, such as a computer program or algorithm, to enable one or more method steps or operations, as disclosed herein. Such computer programs or algorithms can be run automatically or on-demand based on one or more inputs from the users. The user portal and/or a user platform may allow users to connect with each other via the portal or platform, such as for data exchange, thereby forming a network of connected users. Such data exchange can be secure and/or cloud-based. The users may each have an account for accessing the network and utilizing the functions associated with data exchange securely and conveniently. The portal and/or platform may include a user interface, e.g., graphical user interface (GUI). The portal and/or platform may include a web application or mobile application. The portal and/or platform may include a digital display to display information to the user and/or an input device that can interact with the user to accept input from the user.

In some embodiments, the electronic report comprising data or health regimens are presented on a user interface, such as a graphical user interface (GUI), of an electronic device of a user (e.g., the subject). The electronic report may be transmitted to a remote user (e.g., a clinical practitioner, a nutrigenetics counselor, or a sports coach). Further, the electronic report can be stored on a remote server (e.g., a cloud-based server).

### Classifiers

The profiling method may comprise processing genetic information and environmental information of a subject using a trained algorithm (e.g., a classifier) to determine a performance or health risk state of the subject. The classifier may be used to classify the subject as having a given performance or health risk state. The classifier may comprise a supervised machine learning algorithm or an unsupervised machine learning algorithm. The classifier may comprise a classification and regression tree (CART) algorithm. The classifier may comprise, for example, a support vector machine (SVM), a linear regression, a logistic regression, a nonlinear regression, a neural network, a Random Forest, a deep learning algorithm, a naïve Bayes classifier. The classifier may comprise an unsupervised machine learning algorithm, e.g., clustering analysis (e.g., k-means clustering, hierarchical clustering, mixture models, DBSCAN, OPTICS algorithm), principal component analysis, independent component analysis, non-negative matrix factorization, singular value decomposition, anomaly detection (e.g., local outlier factor), neural network (e.g., autoencoder, deep belief network. Hebbian learning, generative adversarial network, self-organizing map), expectation-maximization algorithm, and method of moments.

The classifier may be configured to accept a plurality of input variables and to produce one or more output values based on the plurality of input variables. The plurality of input variables may comprise genetic information and environmental information. For example, an input variable may comprise a set of identified variants or alleles, and/or a number of sequences corresponding to or aligning to each of the set of identified variants or alleles. For example, an input variable may comprise a set of genes or pathways corresponding to a polygenic risk or pathway score, and/or a number of sequences corresponding to or aligning to each of the set of genes or pathways.

The classifier may have one or more possible output values, each comprising one of a fixed number of possible values (e.g., a linear classifier, a logistic regression classifier, etc.) indicating a classification of the biological sample into a performance or health risk state (e.g., level of impact of an allele on a pathway). The classifier may comprise a binary classifier, such that each of the one or more output values comprises one of two values (e.g., {0, 1}, {positive, negative}, or, {high risk, normal risk}) indicating a classification of the subject into a performance or health risk state (e.g., level of impact). The classifier may be another type of classifier, such that each of the one or more output values comprises one of more than two values (e.g., {0, 1, 2}, {positive, negative, or indeterminate}, or {high risk, normal risk, or indeterminate}) indicating a classification of the subject as having a performance or health risk state. The output values may comprise descriptive labels, numerical values, or a combination thereof. Some of the output values may comprise descriptive labels. Such descriptive labels may provide an identification or indication of the performance or health risk state of the subject. Such descriptive labels may provide an identification of a recommendation for the subject's performance or health risk state, and may comprise, for example, a therapeutic intervention, a duration of the therapeutic intervention, and/or a recommendation related to diet, exercise, sports training, supplements, functional tests, blood tests, brain management, behavior change, skin care, environmental exposure, stress management, and/or mental health. Such descriptive labels may provide an identification of secondary clinical tests that may be appropriate to perform on the subject, and may comprise, for example, a biopsy, a blood test, a functional test, a computed tomography (CT) scan, a magnetic resonance imaging (MRI) scan, an ultrasound scan, a chest X-ray, a positron emission tomography (PET) scan, or a PET-CT scan. Such descriptive labels may provide a prognosis of the disease state of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1 (e.g., of the classification of the subject as having a performance or health risk state). Such continuous output values may comprise, for example, an un-normalized probability value of at least 0. Such continuous output values may comprise, for example, an un-normalized probability value of at least 0. Such continuous output values may comprise, for example, an indication of an expected duration of an intervention. Such continuous output values may comprise, for example, a hazard ratio or odds ratio for a risk (e.g., injury risk or disease risk). Some numerical values may be mapped to descriptive labels, for example, by mapping 1 to “positive” and 0 to “negative.”

Some of the output values may be assigned based on one or more cutoff values. For example, a binary classification may assign an output value of “positive” or 1 if the subject has at least a 50% probability of being recommended an intervention. For example, a binary classification of subjects may assign an output value of “negative” or (if the subject has less than a 50% probability of being recommended an intervention. In this case, a single cutoff value of 50% is used to classify subjects into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 98%, and about 99%.

As another example, a classification of subjects may assign an output value of “positive” or 1 if the subject has a probability of being recommended an intervention of at least 50%, at least about 55%, at least about 60%, at least about 65%, at least about 70%, at least about 75%, at least about 80%, at least about 85%, at least about 90%, at least about 95%, at least about 98%, or at least about 99%. The classification may assign an output value of “positive” or 1 if the subject has a probability of being recommended an intervention of more than 50%, more than 55%, more than 60%, more than 65%, more than 70%, more than 75%, more than 80%, more than 85%, more than 90%, more than 95%, more than 98%, or more than 99%. The classification may assign an output value of “negative impact” or 0 if the subject has a probability of being recommended an intervention of less than 50%, less than 45%, less than 40%, less than 35%, less than 30%, less than 25%, less than 20%, less than 10%, less than 5%, less than 2%, or less than 1%. The classification may assign an output value of “negative” or 0 if the subject has a probability of being recommended an intervention of no more than 50%, no more than 45%, no more than 40%, no more than 35%, no more than 30%, no more than 25%, no more than 20%, no more than 10%, no more than 5%, no more than 2%, or no more than 1%. The classification may assign an output value of “indeterminate” or 2 if the subject has not been classified as “positive.” “negative,” 1, or 0. In this case, a set of two cutoff values is used to classify subjects into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subjects into one of n+1 possible output values, where n is any positive integer.

The classifier may be trained with a plurality of independent training samples. Each of the independent training samples may comprise a subject, associated data, and one or more known output values corresponding to performance or health risk states of the subject. Independent training samples may comprise data and outputs obtained from a plurality of different subjects. Independent training samples may comprise data and outputs obtained at a plurality of different time points from the same subject. Independent training samples may be associated with presence of a performance or health risk state (e.g., training samples obtained from a plurality of subjects known to have the performance or health risk state). Independent training samples may be associated with absence of a performance or health risk state (e.g., training samples obtained from a plurality of subjects who are known to not have the performance or health risk state).

The classifier may be trained with at least about 50, at least about 100, at least about 150, at least about 200, at least about 250, at least about 300, at least about 350, at least about 400, at least about 450, or at least about 500 independent training samples. The independent training samples may comprise subjects associated with presence of the performance or health risk state and/or subjects associated with absence of the performance or health risk state. The classifier may be trained with no more than 500, no more than 450, no more than 400, no more than 350, no more than 300, no more than 250, no more than 200, no more than 150, no more than 100, or no more than 50 independent training samples associated with presence of the performance or health risk state.

The classifier may be trained with a first number of independent training samples associated with a presence of the performance or health risk state and a second number of independent training samples associated with an absence of the performance or health risk state. The first number of independent training samples associated with a presence of the performance or health risk state may be no more than the second number of independent training samples associated with an absence of the performance or health risk state. The first number of independent training samples associated with a presence of the performance or health risk state may be equal to the second number of independent training samples associated with an absence of the performance or health risk state. The first number of independent training samples associated with a presence of the performance or health risk state may be greater than the second number of independent training samples associated with an absence of the performance or health risk state.

The classifier may be configured to identify the performance or health risk state with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%; for at least about 50, at least about 100, at least about 150, at least about 200, at least about 250, at least about 300, or more than about 300 independent samples. The accuracy of identifying the performance or health risk state by the classifier may be calculated as the percentage of independent test samples (e.g., subjects having the performance or health risk state) that are correctly identified or classified as having or not having the performance or health risk state, respectively.

The classifier may be configured to identify the performance or health risk state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The PPV of identifying the performance or health risk state by the classifier may be calculated as the percentage of biological samples identified or classified as having the performance or health risk state that correspond to subjects that truly have the performance or health risk state. A PPV may also be referred to as a precision.

The classifier may be configured to identify the performance or health risk state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The NPV of identifying the performance or health risk state by the classifier may be calculated as the percentage of biological samples identified or classified as not having the performance or health risk state that correspond to subjects that truly do not have the performance or health risk state.

The classifier may be configured to identify the performance or health risk state with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical sensitivity of identifying the performance or health risk state by the classifier may be calculated as the percentage of independent test samples associated with presence of the performance or health risk state (e.g., subjects known to have the performance or health risk state) that are correctly identified or classified as having the performance or health risk state. A clinical sensitivity may also be referred to as a recall.

The classifier may be configured to identify the performance or health risk state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The clinical specificity of identifying the performance or health risk state by the classifier may be calculated as the percentage of independent test samples associated with absence of the performance or health risk state (e.g., apparently healthy subjects with negative clinical test results for the performance or health risk state) that are correctly identified or classified as not having the performance or health risk state.

The classifier may be configured to identify the performance or health risk state with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The AUC may be calculated as an integral of the Receiver Operator Characteristic (ROC) curve (e.g., the area under the ROC curve) associated with the classifier in classifying biological samples as having or not having the performance or health risk state.

The classifier may be adjusted or tuned to improve the performance, accuracy, PPV, NPV, clinical sensitivity, clinical specificity, or AUC of identifying one or more performance or health risk states. The classifier may be adjusted or tuned by adjusting parameters of the classifier (e.g., a set of cutoff values used to classify a sample as described elsewhere herein, or weights of a neural network). The classifier may be adjusted or tuned continuously during the training process or after the training process has completed.

After the classifier is initially trained, a subset of the inputs may be identified as most influential or most important to be included for making high-quality classifications. For example, a subset of the input data may be identified as most influential or most important to be included for making high-quality classifications or identifications of the performance or health risk state. The set of input data or a subset thereof may be ranked based on metrics indicative of each feature's influence or importance toward making high-quality classifications or identifications of the performance or health risk state. Such metrics may be used to reduce, in some cases significantly, the number of input variables (e.g., predictor variables) that may be used to train the classifier to a desired performance level (e.g., based on a desired minimum accuracy, PPV, NPV, clinical sensitivity, clinical specificity, or AUC).

For example, if training the training algorithm with a plurality comprising several dozen or hundreds of input variables in the classifier results in an accuracy of classification of more than 99%, then training the training algorithm instead with only a selected subset of 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, or no more than about 100 such most influential or most important input variables among the plurality results in decreased but still acceptable accuracy of classification (e.g., at least about 70%, at least about 75%, at least about 80%, at least about 85%, at least about 90%, at least about 95%, at least about 96%, at least about 97%, or at least about 98%).

In some embodiments, the subset may be selected by rank-ordering the entire plurality of input variables and selecting a predetermined number (e.g., 1, 2, 3, 4, 5, 6, 7, 8, 9, 10, no more than about 10, no more than about 15, no more than about 20, no more than about 25, no more than about 30, no more than about 35, no more than about 40, no more than about 45, no more than about 50, no more than about 100, no more than about 150, or no more than about 200) of input variables with the best metrics.

### Computer Systems

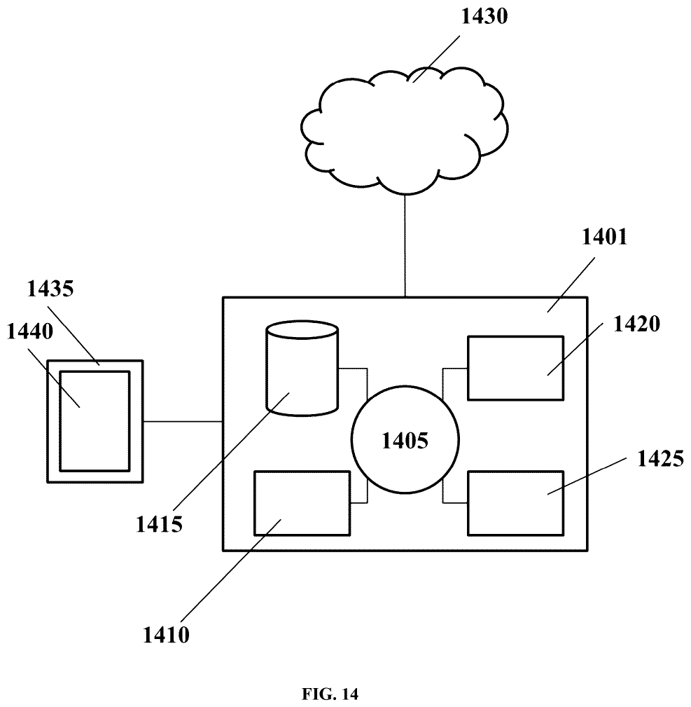

The present disclosure provides computer systems that are programmed to implement methods of the disclosure. FIG. 14 shows a computer system 1401 that is programmed or otherwise configured to perform one or more functions or operations of the present disclosure, such as, for example, determining a performance or health risk state or score, and facilitating nutrigenomics reporting for a subject. The computer system 1401 can regulate various aspects of the portal and/or platform of the present disclosure, such as, for example, receiving genetic information and/or environmental information of a subject, determining a performance or health risk state of the subject, outputting an electronic report (e.g., together with a range of daily scores relating to health, fitness, and sports performance), transmitting an electronic report to a remote user, storing an electronic report on a remote server, and processing input data using a trained algorithm to identify performance or health risk states. The computer system 1401 can be an electronic device of a user or a computer system that is remotely located with respect to the electronic device. The electronic device can be a mobile electronic device.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet.

The network 1430 in some cases is a telecommunication and/or data network. The network 1430 can include one or more computer servers, which can enable distributed computing, such as cloud computing. For example, one or more computer servers may enable cloud computing over the network 1430 (“the cloud”) to perform various aspects of analysis, calculation, and generation of the present disclosure, such as, for example, receiving genetic information and/or environmental information of a subject, determining a performance or health risk state of the subject, outputting an electronic report, transmitting an electronic report to a remote user, storing an electronic report on a remote server, and processing input data using a trained algorithm to identify performance or health risk states. Such cloud computing may be provided by cloud computing platforms such as, for example. Amazon Web Services (AWS). Microsoft Azure. Google Cloud Platform, and IBM cloud. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC). The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., a mobile device of the user). Examples of remote computer systems include personal computers (e.g., portable PC), slate or tablet PC's (e.g., Apple® iPad, Samsung® Galaxy Tab), telephones. Smart phones (e.g., Apple® iPhone, Android-enabled device, Blackberry®), or personal digital assistants. The user can access the computer system 1401 via the network 1430.

Methods provided herein can be implemented by way of machine (e.g., computer processor) executable code stored on an electronic storage location of the computer system 1401, such as, for example, on the memory 1410 or electronic storage unit 1415. The machine-executable or machine-readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc, shown in the drawings. Volatile storage media include dynamic memory, such as main memory of such a computer platform. Tangible transmission media include coaxial cables: copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM. DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

The computer system 1401 can include or be in communication with an electronic display 1435 that comprises a user interface (UI) 1440 for providing, for example, genomic or other data management. Examples of user interfaces include, without limitation, a graphical user interface (GUI) and web-based user interface. The user interface may be provided via an application programming interface (API).

Methods and systems of the present disclosure can be implemented by way of one or more algorithms. An algorithm can be implemented by way of software upon execution by the central processing unit 1405. The algorithm can, for example, receive genetic information and/or environmental information of a subject, determine a performance or health risk state of the subject, outputting an electronic report, transmit an electronic report to a remote user, store an electronic report on a remote server, and process input data using a trained algorithm to identify performance or health risk states.

### EXAMPLES

### Example 1: Genetic Modifier Algorithms for Calculating Real-Time Moving Scores Related to Fitness and Sports Performance and Athlete Health

Using systems and methods of the present disclosure, genetic modifier algorithms were developed for calculating real-time moving scores related to fitness and sports performance, as well as athlete health. Polygenic pathway scores, together with physiological and performance data obtained from wearable devices, contextual information and subjective feedback, were used as input data to construct and modify physiological models of sports performance, fitness status and athlete health. This results in more accurate and personalized data, scores, estimations, and predictions of health, fitness, and sports performance metrics. Scores are outputted (e.g., displayed to a user via a software user interface) in a digital, easy-to-interpret way, coupled with personal recommendations for the user to improve their physical performance and health, and to reduce risk of injury.

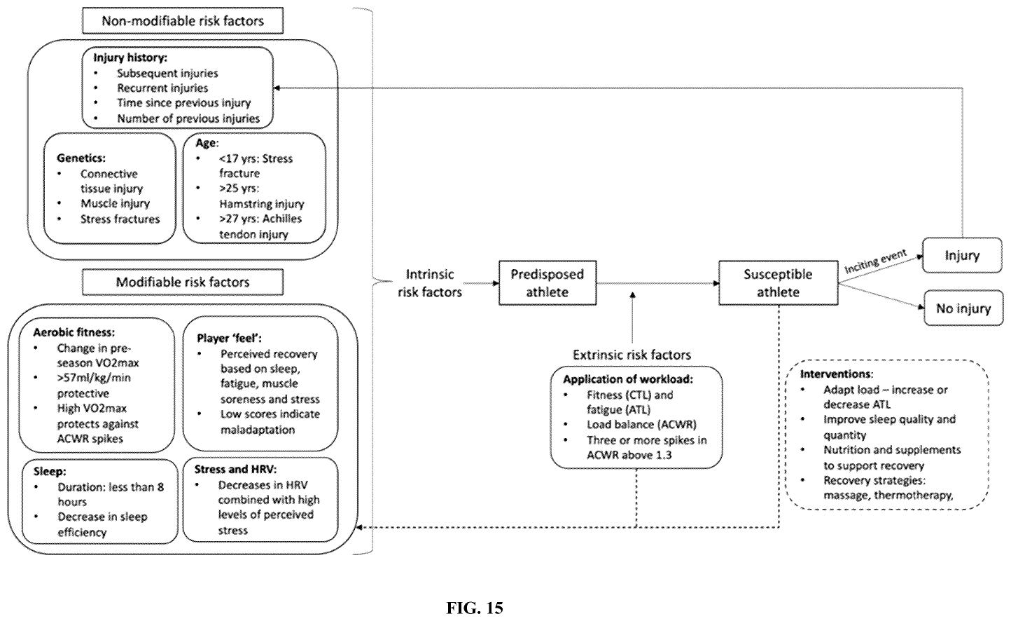

Injury and load balance modifier algorithms and machine learning models were developed as follows. The modifier algorithms generate daily scores for connective tissue injury, muscle injury, stress fractures, rotator cuff injury, anterior cruciate ligament (ACL) injury. Achilles injury, knee osteoarthritis, combined injury and training load balance. FIG. 15 shows an example of a framework for modelling injuries using genetic modifiers.

Polygenic pathway scores were calculated for each of the listed injury types, from SNP genotyping data. Pathway scores were used to modify an acute-to-chronic workload ratio (ACWR) model to account for an athlete's genetic predisposition for different injury types. Features were built using combined genetic modifier scores and an ACWR model, to serve as validation input for statistical models. A time-to-event model was implemented in the form of a multi-state model (MSM) to incorporate an athlete's time-varying training load exposures prior to a non-contact soft tissue injury. The implemented MSM was evaluated with hazard ratios to validate that injury features showed an increased risk for an athlete to sustain a non-contact injury. The input datapoints to this model, excluding the injury features, were obtained from wearable devices. These devices provided an athlete's (i) internal training load measurements (e.g., heart rate) and (ii) external training load measurements (e.g., global positioning system (GPS) data) during training and competition. A machine learning model was also developed to use time-series data with specific features to determine a likelihood of risk of injury. Input features used to develop the machine learning model include stress, sleep, resting heart rate, pathway scores, heritability, CTL, and ATL.

The load balance modifier algorithm used ACWR as adapted by power and endurance pathways. It was interpreted in the context of the injury scores provided digitally. The load balance was normalized to values from 0 to 1, using ranges associated with optimizing training load and performance.

A team score was calculated by combining individual players' health and performance scores. The health and performance scores were calculated from the combination of wearable data, contextual data, and genetic inputs. The combined team score assists managerial staff toward tactical and strategic decision making.

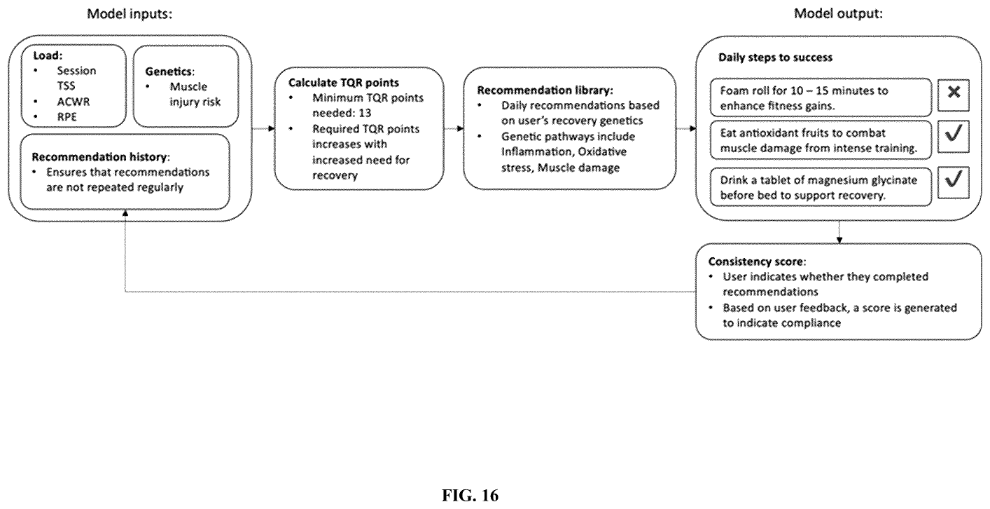

A recovery modifier algorithm was developed as follows. The recovery modifier algorithm generates a daily recovery score, together with actionable nutritional, lifestyle, and supplementation recommendations based on a modified total quality recovery (TQR) model. Polygenic pathway scores for inflammation, oxidative stress, and muscle injury risk, and a combined recovery pathway score were calculated to enable daily personalized, genetic-based recovery recommendations, coupled to a moving (e.g., real-time varying) recovery score. Recommendations were coupled with electronic user feedback for contextual information gathering, that serves as input to the recovery modifier algorithm. FIG. 16 shows an example of a modified TQR algorithm.

Endurance and power modifier algorithms were developed as follows. Polygenic pathway scores for endurance pathways (e.g., VO2 max trainability and slow twitch fibers) and power pathways (e.g., fast twitch fibers, strength, and muscle power) were used as input features to determine dynamic fitness recommendations, using training load data as input data. Recommendations were structured as personalized training plans delivered automatically through a mobile application, and provided individualized aerobic vs anaerobic training volumes to improve fitness levels in an efficient and safe way. A machine learning algorithm was constructed to determine what type of exercises may be more efficient to gain fitness according to an individual's genetics. It also informs training time periods according to an individual's training response.

Two systems were constructed based on use cases, on which the modifier algorithms were executed. The first system was a mobile application for individual athletes, which provided daily fitness, sports performance, and health recommendations, which were determined based on daily scores calculated from specific combinations of genetic, wearable, and contextual data.

The second system was an athlete optimization platform, focused on sports teams, comprising 1.) a desktop application for coaches, medical staff, sports nutritionists, and physical conditioning staff, and 2.) a mobile application for each team member. The desktop application comprised a database of all team members, their genetic data, contextual information, injury history, and real-time data integration with health, fitness, and sports performance hardware, such wearable devices. Modifier scores related to athlete health, fitness, and sports performance were calculated for each team member on a daily basis, with coaching, health, and nutritional recommendations generated and provided to staff members. Combined team scores were also calculated to provide insights on team selection health and performance status. Team members' genetic-only data was not visible to any of the coaching staff, and was stored in a secure HIPAA and POPI compliant manner on the backend.

Team members received their performance and health data through a mobile application, with functionality to capture contextual information and subjective feedback, as well as to provide individual recommendations specific to a team member.

Athlete scores and time series information were presented in an easy-to-interpret visual way, with a red-amber-green status to indicate risk and/or intervention required.

Tables 1-4 illustrate examples of various pathway genes and variants, including injury pathway genes and variants (Table 1), recovery pathway genes and variants (Table 2), endurance pathway genes and variants (Table 3), and power pathway genes and variants (Table 4).

TABLE 1
Injury pathway genes and variants

| Injury pathway | Gene | Variant |
|---|---|---|
| Connective tissue injury | GDF5 | 5′UTR C > T |
| Connective tissue injury | COL1A1 | Sp1 G > T |
| Connective tissue injury | COL3A1 | Ala698Thr G > A |
| Connective tissue injury | MMP12 | 82 A > G |
| Connective tissue injury | COL12A1 | AluI A > G |
| Connective tissue injury | MMP3 | A > G |
| Connective tissue injury | VEGFA | −2578 C > A |
| Connective tissue injury | CASP8 | −652 6N Ins/Del |
| Muscle injury | MMP3 | A > G |
| Muscle injury | ACTN3 | 577 R/X |
| Muscle injury | ACE | Ins/Del |
| Muscle injury | IGF2 | ApaI G > A |
| Stress fractures | COL1A1 | Sp1 G > T |
| Stress fractures | VDR | Taq1 T > C |
| Stress fractures | P2X7R | Ala348Thr G > A |
| Stress fractures | VDR | Fok1 T > C |
| Stress fractures | RANKL | T > C |
| Rotator cuff injury | MMP1 | −1607 1G/2G |
| Rotator cuff injury | SAP30BP | G > A |
| Rotator cuff injury | IL-6 | −174 G > C |
| ACL injury | GDF5 | 5′ UTR C > T |
| ACL injury | COL1A1 | 1546 G > T |
| ACL injury | COL3A1 | Ala698Thr G > A |
| ACL injury | MMP12 | 82 A > G |
| ACL injury | COL12A1 | AluI A > G |
| Achilles injury | GDF5 | 5′ UTR C > T |
| Achilles injury | MMP3 | A > G |
| Achilles injury | VEGFA | −2578 C > A |
| Achilles injury | CASP8 | −652 6N Ins/Del |
| Knee osteoarthritis | GDF5 | 5′UTR C > T |
| Knee osteoarthritis | ESR1 | XbaI A > G |
| Knee osteoarthritis | VDR | Taq1 T > C |
| Knee osteoarthritis | ACE | INS/DEL |

TABLE 2
Recovery pathway genes and variants

| Recovery pathway | Gene | Variant |
|---|---|---|
| Combined recovery | IL-6 | −174 G > C |
| Combined recovery | IL-6R | 48867 A > C |
| Combined recovery | TNFA | −308 G > A |
| Combined recovery | CRP | 2147 G > A |
| Combined recovery | MNSOD | Val16Ala T > C |
| Combined recovery | CAT | −262 C > T |
| Combined recovery | GPX1 | C > T |
| Combined recovery | PPARGC1A | Gly482Ser G > A |
| Combined recovery | ACTN3 | 577 R/X |
| Combined recovery | AMPD1 | 133 C > T |
| Combined recovery | MLCK | 37885 C > A |
| Combined recovery | ACE | INS/DEL |
| Combined recovery | IGF2 | ApaI G > A |
| Recovery Inflammation | IL-6 | −174 G > C |
| Recovery Inflammation | IL-6R | 48867 A > C |
| Recovery Inflammation | TNFA | −308 G > A |
| Recovery Inflammation | CRP | 2147 G > A |
| Recovery oxidative stress | MNSOD | Val16Ala T > C |
| Recovery oxidative stress | CAT | −262 C > T |
| Recovery oxidative stress | GPX1 | C > T |
| Recovery oxidative stress | PPARGC1A | Gly482Ser G > A |
| Recovery muscle damage | ACTN3 | 577 R/X |
| Recovery muscle damage | AMPD1 | 133 C > T |
| Recovery muscle damage | MLCK | 37885 C > A |
| Recovery muscle damage | ACE | INS/DEL |
| Recovery muscle damage | IGF2 | ApaI G > A |

TABLE 3
Endurance pathways genes and variants

| Endurance pathway | Gene | Variant |
|---|---|---|
| VO2max trainability | AMPD1 | 133 C > T |
| VO2max trainability | PPARGC1A | Gly482Ser G > A |
| VO2max trainability | GSTP1 | Ile105Val 313 A > G |
| VO2max trainability | VEGF | −634 G > C |
| VO2max trainability | CKM | Ncol T > C |
| VO2max trainability | HIF1A | Pro582Ser 1744C > T |
| VO2max trainability | ACE | INS/DEL |
| VO2max trainability | CAT | −262 C > T |
| VO2max trainability | NRF2 | A > G |
| VO2max trainability | ACSL1 | T > C |
| Slow twitch fibers | AGTR2 | A > C |
| Slow twitch fibers | ACE | INS/DEL |
| Slow twitch fibers | PPARA | G > C |
| Slow twitch fibers | ACTN3 | 577 R/X |
| Slow twitch fibers | VEGFR | His472Gln T > A |

TABLE 4
Power pathway genes and variants

| Power pathway | Gene | Variant |
|---|---|---|
| Fast twitch fibers | ACTN3 | 577 R/X |
| Fast twitch fibers | HIF1A | Pro582Ser C > T |
| Fast twitch fibers | AGTR2 | A > C |
| Fast twitch fibers | PPARA | 89204 G > C |
| Muscle strength | ACTN3 | 577 R/X |
| Muscle strength | ACE | Ins/Del |
| Muscle strength | HIF1A | Pro582Ser C > T |
| Muscle strength | AGT | Met268Thr A > G |
| Muscle strength | VDR | Bsm1 G > A |
| Muscle strength | VDR | Taq1 T > C |
| Muscle strength | VDR | Fok1 T > C |
| Muscle strength | AMPD1 | 133 C > T |
| Muscle strength | PPARA | 89204 G > C |
| Muscle strength | PPARG | Pro12Ala C > G |
| Muscle strength | ADRB2 | Arg16Gly A > G |
| Muscle power | ACTN3 | 577 R/X |
| Muscle power | ACE | Ins/Del |
| Muscle power | AGT | Met268Thr A > G |
| Muscle power | AMPD1 | 133 C > T |
| Muscle power | PPARA | 89204 G > C |
| Muscle power | PPARG | Pro12Ala C > G |
| Muscle power | NOS3 | −786 T > C |
| Muscle power | IL-6 | −174 G > C |
| Muscle power | ACVR1B | A > G |
| Muscle power | CKM | Ncol T > C |
| Muscle power | ADRB2 | Arg16Gly A > G |
| Muscle power | MCT1 | Glu490Asp A > T |
| Muscle power | MNSOD | Val16Ala T > C |

### Example 2: Integrating Genetic Predisposition into Injury Risk Prevention Tools for Athletes

Using systems and methods of the present disclosure, genetic predisposition was integrated into injury risk prevention tools for athletes. Genetics plays a significant role in injury susceptibility, and yet current approaches may not apply individual genetic predisposition dynamically to athletes' training programs. By continually modifying workload management based on genetic predisposition for injuries, training can become more personalized for each athlete, to help them reach their training goals without compromising their safety.

An injury risk model was developed to expand upon and address certain limitations of the ACWR model of workload and injury management, due to evidence that shows the injury risk model achieves a 17% higher sensitivity for identifying injury risk.

The injury risk model may be applied as a tool to guide athletes to train in a way that is sustainable according to their own genetics, in combination with their current fitness levels and training history. It discourages overtraining and encourages athletes to build towards their goals in a way that avoids spikes in workload, which may increase their likelihood of sustaining an injury they are genetically predisposed to. It supports athletes in reaching their fitness goals and becoming more resilient to the demands of their sport.

The implications of the injury risk tool highlight a simple message—workload management and injury prevention cannot have a one-size-fits-all approach. The injury risk tool allows athletes and coaches to make informed training decisions that are most appropriate for each individual, ensuring their goals are reached in a way that is optimal for their unique genetic predisposition to injury.

Workload management is important because injuries have a significant negative impact on athletes, lowering their performance [1] and impacting their psychological wellbeing [2]. For sports teams, injuries have a knock-on effect on the entire team's performance. A significant relationship between a reduced injury and improved performance may be observed in football teams, where a lower injury burden was linked to success in UEFA Champions League [3]. Improving injury prevention strategies is inevitably a top priority for athletes, as well as for the coaches, sports scientists, and teams whose primary goal is to support athletes as they strive for peak performance.

In that effort, athletes can be inclined to push themselves to the limit to further refine their skills, increase their fitness and perform at their best in competitions. However, when the body is not given sufficient time to recover and adapt between periods of exertion, athletes are more likely to sustain an overuse injury [4]. Overuse injuries are in fact ubiquitous across the world of sports, accounting for up to 50% of injuries [5]. To prevent an overuse injury, careful monitoring and adjusting of an athlete's workload is needed to strike the right balance between exertion and adaptation [6].

One tool currently utilized to help determine when an athlete may be at risk of crossing the line from effective training to risking injury is the acute-to-chronic workload ratio (ACWR). The ACWR may be used to monitor an athlete's fitness and fatigue, and provides a snapshot view of injury risk at any given point in time. The ACWR may be validated in various sports, including soccer, rugby. Australian football, and cricket [7], and is recommended for workload management and injury prevention by the International Olympic Committee [8]. However, the ACWR's utility for injury prevention has its limitations by not accounting for moderators of workload that increase or decrease injury risk.

Genetic variation may be a significant factor in injuries. A strong moderator of the workload-injury relationship is an athlete's genetic predisposition [9] as it is a significant intrinsic risk factor for injury [10,11]. Genetic variation has an influence on how individuals adapt to training [12,13] and moderates the association between spikes in workload and subsequent injury [14]. Gene variants have an impact on the biochemical pathways that play a role in musculoskeletal function and adaptation to load [12,13]. Family and twin studies have yielded heritability estimates (the extent to which a feature can be attributed to genetics) for injuries ranging from 40-69% for different types of injury [13,15,16], indicating a definitive link between genetic predisposition and injury.

In a study of 289 soccer players, each player was tested for genetic markers associated with injuries, and the soccer players with genetic markers for GDF5, AMPD1, COL5A1, or IGF were more likely to be injured during the season and played significantly fewer matches than players who did not carry these genetic markers [17].

The MMP3 genetic marker is also strongly associated with hamstring injury, with each copy of the genetic marker increasing the risk of hamstring injury two-fold in soccer players [18]. Similar relationships may exist between genetic markers and ACL injuries [19], rotator cuff tears [20], and stress fractures [21]. Therefore, not only does genetics play a general role in injury risk in individuals, but it impacts specific injury sites as well.

Genetics may be integrated into workload management. A limitation of genetic information is that, while it may be useful in identifying athletes that are injury-prone, it only provides part of the story. The injury risk model was developed to dynamically incorporate genetic predispositions into an athlete's workload, along with other stressors, to determine how an athlete's genetics influence their daily performance, as well as measure the extent to which genetics is a moderator in the workload-injury relationship.

By modifying an athlete's daily workload and ACWR according to their genetic predisposition for injuries, the injury risk tool gives an athlete a daily, personalized, and actionable measure of how their current workload, adapted according to their unique genetics, is either increasing or decreasing their injury susceptibility.

To determine the validity to personalize an athlete's training load using genetics, historical training data, workload data, genetic data and injury history was collected from 120 recreational endurance athletes. Machine learning and pathway analysis methodologies were utilized to calculate the integrative impact of multiple genetic markers on susceptibility for specific injuries. Achilles tendon. Hamstring. Knee ligament (ACL). Stress fractures, and Rotator cuff injuries were investigated.

Data was collected from 120 recreational endurance athletes: historical training data, spanning over a period of 18 months up to 120 months; survey data regarding injury history, including date of injury, injury type and severity, which were then categorized into acute and overuse injuries, with a total of 70 overuse injuries included in the final analysis; workload data from global positioning system (GPS) enabled wearable devices, often used alongside electrocardiogram (ECG) chest straps: genetic markers for five types of injury, shown in Table 5.

TABLE 5
Genetic markers for five different types of injuries

| Injury Type | Genetic Markers |
|---|---|
| Achilles tendon injury | GDF5, MMP3, COL5A1, VEGFA |
| Hamstring injury | ACTN3, ACE, IGF2, MMP3 |
| Knee ligament injury (ACL) | GDF5, COL1A1, COL3A1, COL5A1, |
| | MMP12 |
| Stress fractures | COL1A1, VDR Fok1, VDR Taq1, |
| | P2X7R, RANKL |
| Rotator cuff injury | MMP1, MMP3, SAP30BP, IL6 |

Training load was calculated from the participants' training stress score (TSS). TSS is a measure of the workload from a training session. It may be the product of the workout's intensity and duration—as either of these increases, TSS also increases. TSS may be calculated using the following expression:

$$TSS = IF \times {t}_{min}$$

$$IF = \frac{{Effort}_{session}}{{Effort}_{threshold}}$$

where: tₘᵢₙ is a duration of the workout in minutes, IF is an Intensity Factor (how intense the effort was), and threshold is a performance threshold metric (e.g., HR, pace, and/or power) at threshold level.

Chronic training load (CTL) can be considered a measure of an athlete's fitness. It is a moving average of an athlete's daily training load (TSS) over a past duration depending on the sport, such as the last six weeks or 42 days of data points. CTL may be calculated using the following expression:

$$CTL = {TSS}_{Today} \times ( 1 - exp ( \frac{- 1}{TC} ) ) + {CTL}_{Yesterday} \times exp ( \frac{- 1}{TC} )$$

where: TC is a Time Constant (the TC standard may be any duration depending on the sport, such as 42 days for CTL, but can vary according to application).

Acute training load (ATL) relates to how an athlete's most recent training impacts their body, considered as a measure of fatigue. ATL is a moving average of an athlete's TSS over the last 7 days. ATL may be calculated using the following expression:

$$ATL = {TSS}_{Today} \times ( 1 - exp ( \frac{- 1}{TC} ) ) + {CTL}_{Yesterday} \times exp ( \frac{- 1}{TC} )$$

where: TC is a Time Constant (the TC standard is 7 days for ATL but can vary according to application).

The relationship between an athlete's fatigue (ATL) and fitness (CTL) is described by the exponentially weighted moving averages (EWMA) ACWR. A peak ACWR of higher than 1.5 a week before, or within the week of injury, has been shown to be associated with an increased susceptibility for sustaining an injury [22,23]. ACWR may be calculated using the following expression:

$$ACWR = ATL : CTL$$

For both the standard ACWR model and the injury risk model, the association between injury and spikes in workload two weeks before, one week before, and the week of injury were investigated. To compare the two models, a binary classification system was developed, which was trained to classify athletes as sustaining an injury or remaining injury free.

There are four possible outcomes using this classification system: (1) True positive, where the models indicated injury risk and the athlete was injured; (2) True negative, where the models indicated no injury risk and the athlete was not injured; (3) False positive, where the models indicated injury risk, but the athlete avoided injury; and (4) False negative, where the models did not indicate injury risk, but the athlete was injured.

The ability of the injury risk model to identify the risk of sustaining an injury was compared to that of the standard ACWR model. Sensitivity and specificity were used to measure and compare model performance [24].

The results indicated that the injury risk model identifies injuries in athletes with greater sensitivity as compared to the standard ACWR model. The injury risk model correctly identified up to 17% more incidences of injuries in comparison to the standard ACWR model. In addition, the injury risk model has a high specificity of up to 77%, indicating the model correctly indicated when an athlete was not at risk for an injury 77% of the time.

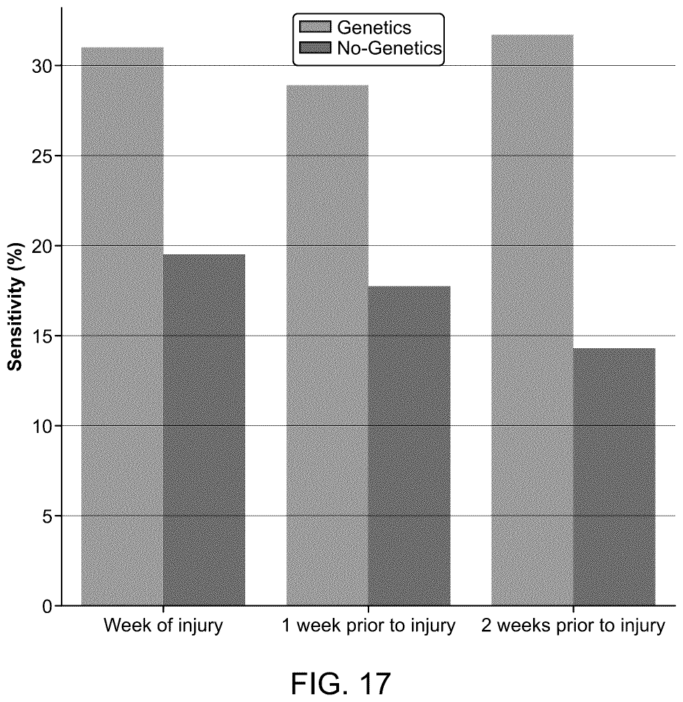

Sensitivity, also known as the true positive rate, represents the proportion of injured athletes that were correctly identified by the models as at risk for injury. As shown in FIG. 17, the injury risk model (red) had an 11%, 11%, and 17% higher sensitivity for spikes in workload in (i) the week of injury: (ii) one week prior to the injury; and (iii) two weeks prior to the injury, respectively, compared to the standard ACWR model (purple).

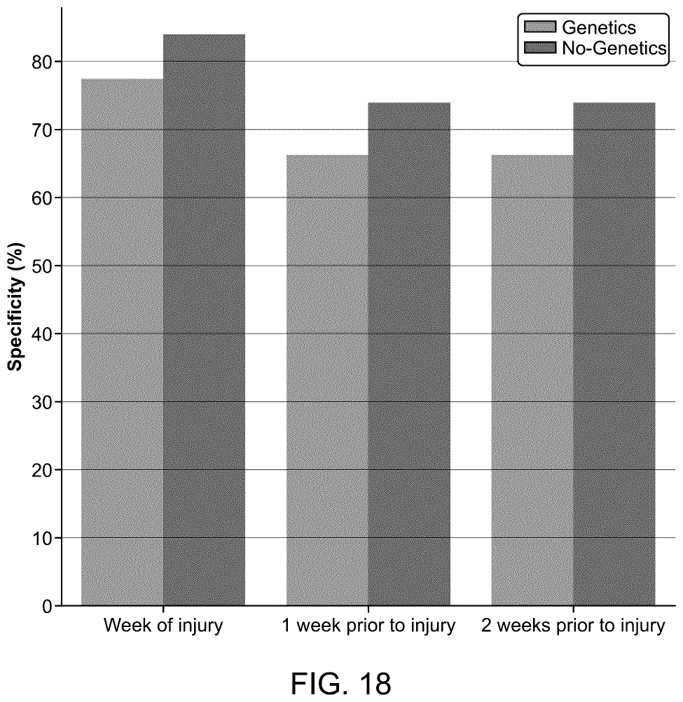

Specificity represents the proportion of uninjured athletes that were correctly classified as not at risk for an injury, also referred to as the true negative rate [24]. Both the injury risk model and standard ACWR model had a high specificity, as shown in FIG. 18. In comparison to the standard ACWR model (purple), the Injury Risk model (red) had a 6%, 8%, and 8% lower specificity for spikes in ACWR in (i) the week of injury: (ii) one week prior to the injury; and (iii) two weeks prior to the injury, respectively.

The odds ratio (OR) of an injury occurring within the week of when the Injury Risk model indicated injury risk, was calculated to be 1.54 (95% Confidence Interval (CI) 0.8-2.95). This indicates that athletes who were identified to be at risk for injury by the injury risk model were 1.54 times more likely to sustain an injury than those who were not identified to be at risk for injury.

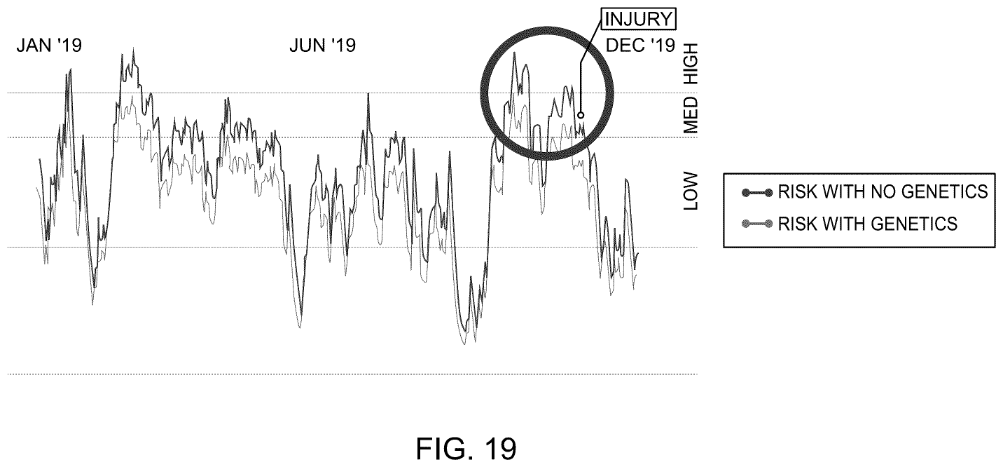

The injury risk model may be applied to an athlete's workload. The time series in FIG. 19 illustrates an example of how the injury risk model more accurately determined an athlete's injury risk in comparison to the standard ACWR model. The graph indicates the athlete's injury risk (low, medium, or high) during a period they were training for multiple ironman races. Throughout this period, the standard ACWR model (purple line) underestimated the athlete's injury risk.

Notably, a week prior to sustaining an Achilles tendon injury, the standard ACWR model failed to indicate that the athlete was at risk of sustaining an injury. In contrast, the injury risk model (red line) accurately indicated that the athlete was at risk of sustaining an injury a week prior to the incidence of the injury.

The results of the injury risk model validation study highlight the significance of the role of genetics in how athletes respond to high training loads. Athletes who carry genetic markers that increase their predisposition for certain injuries are less resilient to sudden spikes in training workload. This is evidenced by the 17% increased sensitivity of the injury risk model to correctly identify when an athlete has a higher risk to become injured in comparison to the standard ACWR model.

In the real-world scenario of an ironman athlete, in cases where the athlete had access to the injury risk tool at the time and took into consideration their genetic predisposition for Achilles tendon injuries, they may have adapted their training plan and avoided the Achilles tendon injury that caused them to stop training for several weeks.

The injury risk model not only provides an indication of whether an athlete is susceptible to injury, but it also represents an invaluable tool for personalized workload management. In its application, the injury risk tool provides an athlete with a daily indication of whether their current training load is optimal, based on their current fitness and their genetic predisposition for injury.

An important principle in the application of the injury risk tool is that the optimal training plan may be based on the gradual progression of workload, considering the genetic predisposition the athlete has for specific injuries. By increasing fitness in a sustainable way, and considering training history, the tool recommends the best actions the athlete can take to become more resilient to the types of injuries they are genetically predisposed to and protect them from spikes in their workload, which may incite injuries.

Athletes using the injury risk tool for their training are encouraged to take into account how their unique genetic makeup impacts their workload strategies. This deeper layer of personalization may bring with it the peace of mind that their injury risk is being more precisely monitored and provide an additional layer of confidence when training and competing.

Whether applying the injury risk tool for use with elite athletes, professional sports teams or for personal fitness goals, an objective is to achieve, for each individual, optimized training with minimal risk of injuries or even zero injuries.

### REFERENCES

[1] G. Verrall, Y. Kalairajah, J. Slavotinek and A. Spriggins, “Assessment of player performance following return to sport after hamstring muscle strain injury.,” Journal of Science and Medicine in Sport, pp. 87-90, 2006 is incorporated by reference herein in its entirety.
[2] L. Podlog and R. Eklund. “Return to sport after serious injury: a retrospective examination of motivation and psychological outcomes.,” Journal of sport rehabilitation, pp. 20-34, 2005 is incorporated by reference herein in its entirety.
[3] M. Hägglund, M. Walden, H. Magnusson, K. Kristenson, H. Bengtsson and J. Ekstrand, “Injuries affect team performance negatively in professional football: an 11-year follow-up of the UEFA Champions League injury study.,” British journal of sports medicine, pp. 738-742, 2013 is incorporated by reference herein in its entirety.
[4] J. Yang, A. Tibbetts, T. Covassin, G. Cheng, S. Nayar and E. Heiden, “Epidemiology of overuse and acute injuries among competitive collegiate athletes.,” Journal of athletic training, pp. 198-204, 2012 is incorporated by reference herein in its entirety.
[5] M. Smucny, S. Parikh and N. Pandya. “Consequences of single sport specialization in the pediatric and adolescent athlete.,” Orthopedic Clinics, vol. 46, no. 2, pp. 249-258, 2015 is incorporated by reference herein in its entirety.
[6] R. Morton, “Modelling training and overtraining.,” Journal of sports sciences, pp. 335-340, 1997 is incorporated by reference herein in its entirety.
[7] R. Andrade, E. Wik, A. Rebelo-Marques, P. Blanch, R. Whiteley, J. Espregueira-Mendes and T. Gabbett, “Is the acute: chronic workload ratio (ACWR) associated with risk of Time-Loss injury in professional team sports? A systematic review of methodology, variables and injury risk in practical situations.,” Sports medicine, pp. 1613-1635, 2020 is incorporated by reference herein in its entirety.
[8] T. Soligard. M. Schwellnus. J. Alonso. R. Bahr. B. Clarsen. Dijkstra. G. T. H. P., M. Gleeson. M. Hägglund. M. Hutchinson and C. van Rensburg. “How much is too much? (Part 1) International Olympic Committee consensus statement on load in sport and risk of injury.,” British journal of sports medicine, pp. 1030-1041, 2016 is incorporated by reference herein in its entirety.
[9] T. Gabbett. “Debunking the myths about training load, injury and performance: empirical evidence, hot topics and recommendations for practitioners.,” British journal of sports medicine, pp. 58-66, 2020 is incorporated by reference herein in its entirety.
[10] T. Lim. C. Santiago. H. Pareja-Galeano. T. Iturriaga. A. Sosa-Pedreschi. N. Fuku. M. Pérez-Ruiz and T. Yvert. “Genetic variations associated with non-contact muscle injuries in sport: A systematic review.,” Scandinavian Journal of Medicine & Science in Sports. pp. 2014-2032., 2021 is incorporated by reference herein in its entirety.
[11] N. Vaughn. H. Stepanyan. R. Gallo and A. Dhawan. “Genetic factors in tendon injury: a systematic review of the literature.,” Orthopaedic journal of sports medicine, p. p.2325967117724416, 2017 is incorporated by reference herein in its entirety.
[12] R. Tashjian. A. Hollins. H. Kim. S. Teefey. W. Middleton. K. Steger-May, L. Galatz and Y. Yamaguchi. “Factors affecting healing rates after arthroscopic double-row rotator cuff repair.,” American Journal of Sports Medicine, p. 2435-42, 2010 is incorporated by reference herein in its entirety.
[13] K. Magnusson. A. Turkiewicz. R. Frobell and M. Englund. “High genetic contribution to anterior cruciate ligament rupture: Heritability ˜69%.” British journal of sports medicine, pp. 385-389, 2021 is incorporated by reference herein in its entirety.
[14] J. Windt and T. Gabbett. “How do training and competition workloads relate to injury? The workload-injury aetiology model.,” British Journal of Sports Medicine, vol. 51. no. 5, pp. 428-435, 2017 is incorporated by reference herein in its entirety.
[15] T. Andrew. L. Antioniades. K. Scurrah. A. MacGregor and T. Spector. “Risk of wrist fracture in women is heritable and is influenced by genes that are largely independent of those influencing BMD.,” Journal of bone and mineral research, pp. 67-74, 2005 is incorporated by reference herein in its entirety.
[16] A. Hakim. L. Cherkas. T. Spector and A. MacGregor. “Genetic associations between frozen shoulder and tennis elbow: a female twin study.,” Rheumatology, pp. 739-742, 2003 is incorporated by reference herein in its entirety.
[17] K. McCabe and C. Collins. “Can genetics predict sports injury? The association of the genes GDF5, AMPD1, COL5A1 and IGF2 on soccer player injury occurrence.,” Sports, p. 21, 2018 is incorporated by reference herein in its entirety.
[18] J. Larruskain, D. Celorrio, I. Barrio, A. Odriozola, S. Gil, J. Fernandez-Lopez, R. Nozal, I. Ortuzar, J. Lekue and J. Aznar, “Genetic Variants and Hamstring Injury in Soccer: An Association and Validation Study.,” Medicine and science in sports and exercise, pp. 361-368, 2018 is incorporated by reference herein in its entirety.
[19] M. Rahim, A. Gibbon, H. Hobbs, W. van der Merwe, M. Posthumus, M. Collins and A. September, “The association of genes involved in the angiogenesis-associated signaling pathway with risk of anterior cruciate ligament rupture.,” Journal of Orthopaedic Research, pp. 1612-1618, 2014 is incorporated by reference herein in its entirety.
[20] J. Assunção, A. Godoy-Santos, M. Dos Santos, E. Malavolta, M. Gracitelli and A. Neto, “Matrix metalloproteases 1 and 3 promoter gene polymorphism is associated with rotator cuff tear.,” Clinical Orthopaedics and Related Research, pp. 1904-1910., 2017 is incorporated by reference herein in its entirety.
[21] I. Varley, D. Hughes, J. Greeves, T. Stellingwerff, C. Ranson, W. Fraser and C. Sale, “RANK/RANKL/OPG pathway: genetic associations with stress fracture period prevalence in elite athletes.,” Bone, pp. 131-136, 2015 is incorporated by reference herein in its entirety.
[22] B. Hulin, T. Gabbett, D. Lawson, P. Caputi and J. Sampson, “The acute: chronic workload ratio predicts injury: high chronic workload may decrease injury risk in elite rugby league players.” British Journal of Sports Medicine, pp. 231-236, 2015 is incorporated by reference herein in its entirety.
[23] T. Gabbett, “The training-injury prevention paradox: should athletes be training smarter and harder?,” British journal of sports medicine, pp. 273-280, 2016 is incorporated by reference herein in its entirety.
[24] J. Ruddy. S. Cormack, R. Whiteley, M. Williams, R. Timmins and D. Opar, “Modeling the risk of team sport injuries: a narrative review of different statistical approaches.,” Frontiers in physiology, vol. 10, p. 829, 2019 is incorporated by reference herein in its entirety.

### Example 3: Software Application User Interface

Using systems and methods of the present disclosure, a software application user interface was developed to be used by various users, including a player (e.g., subject) and a manager.

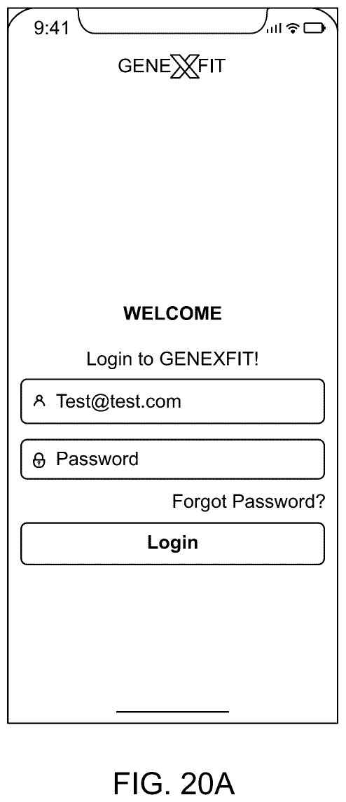

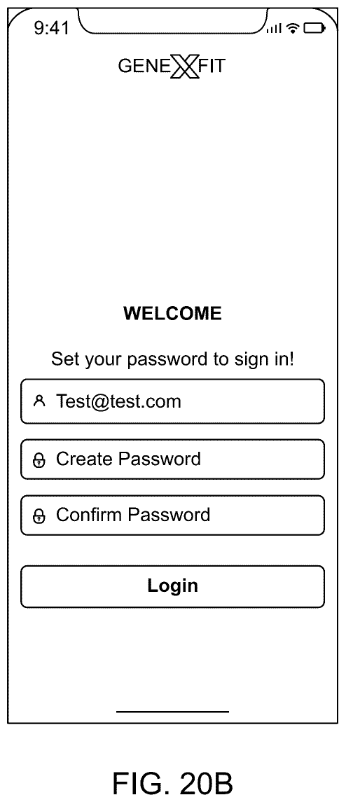

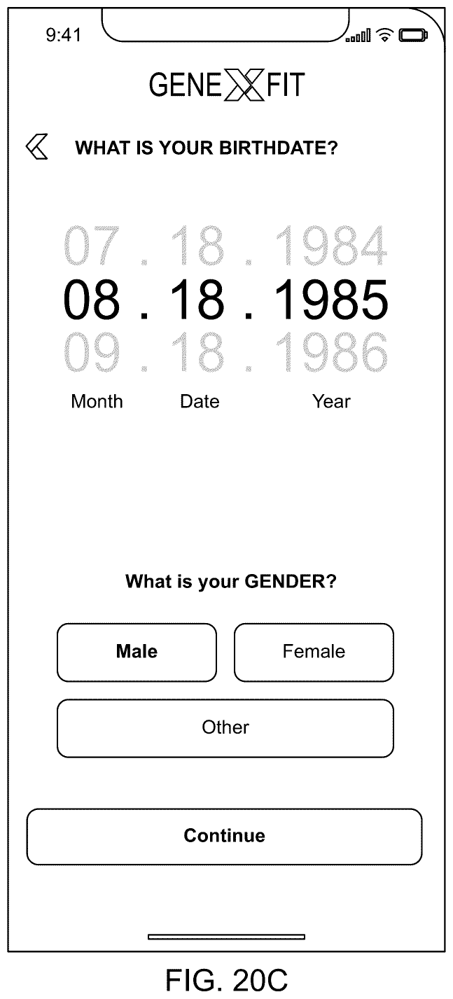

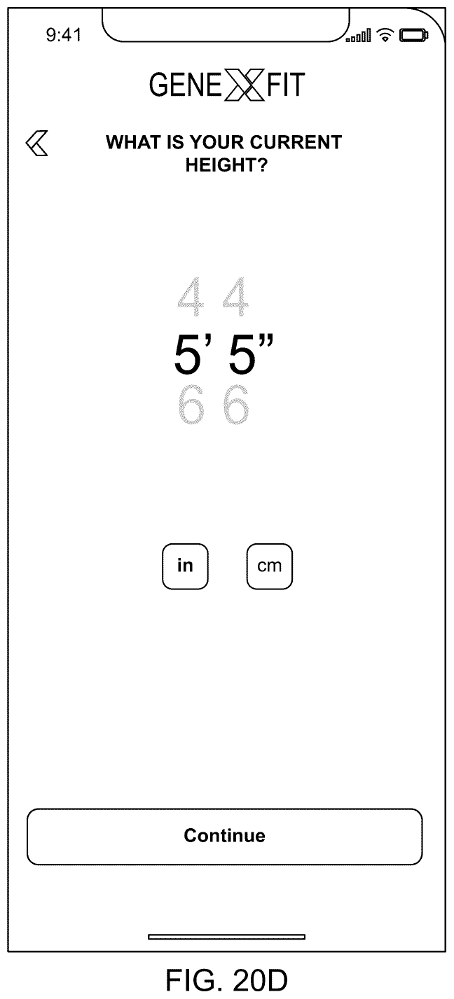

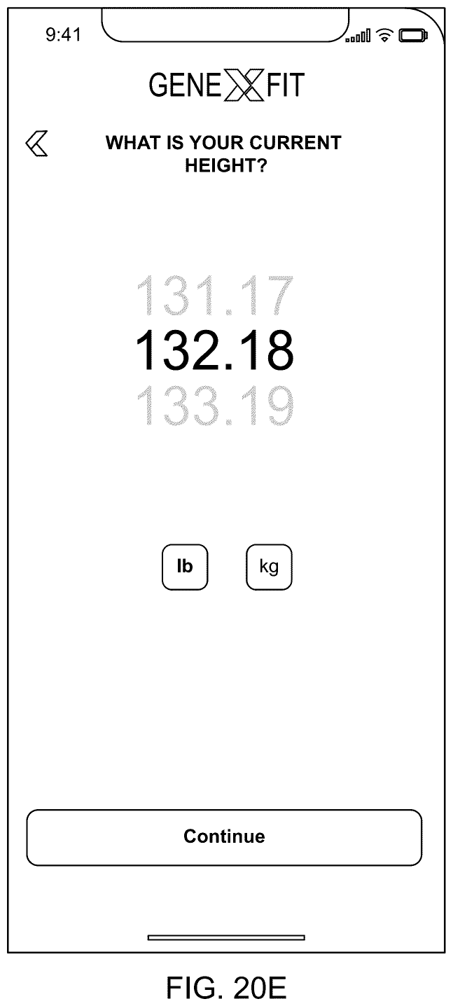

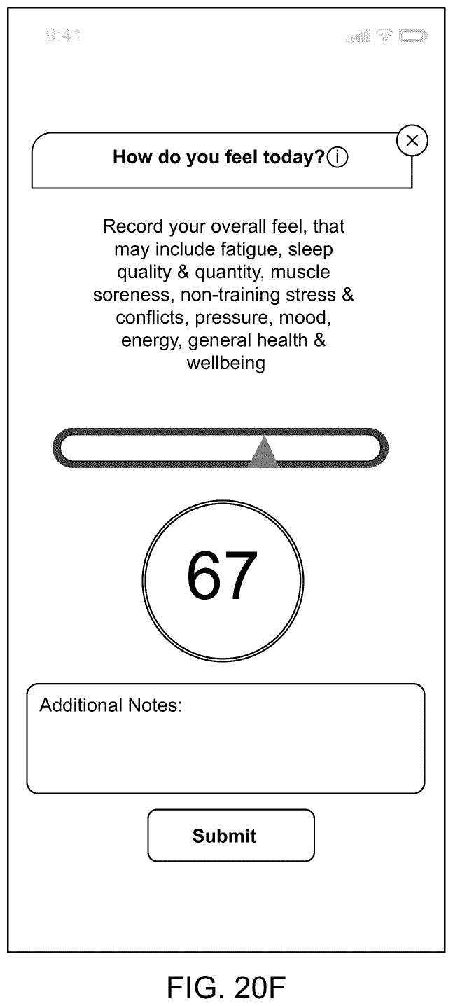

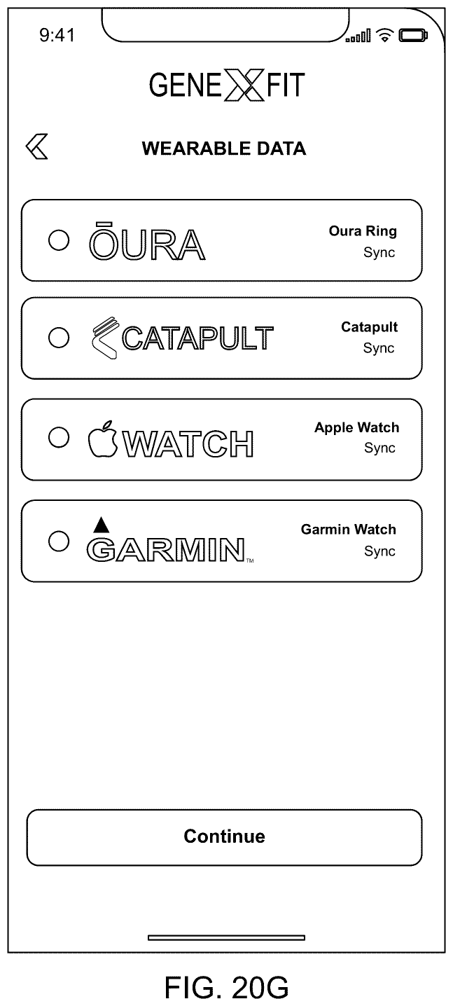

FIGS. 20A-20G show examples of various views of a software application user interface from a user perspective of a player (e.g., subject).

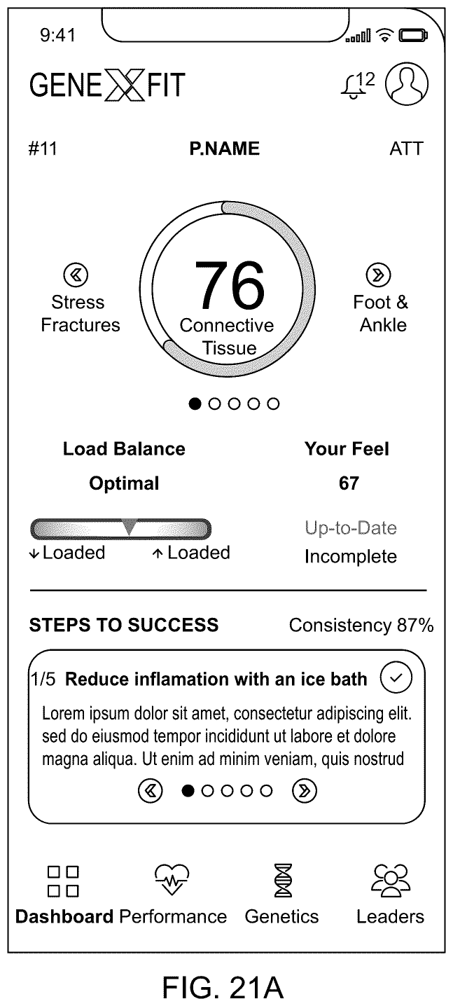

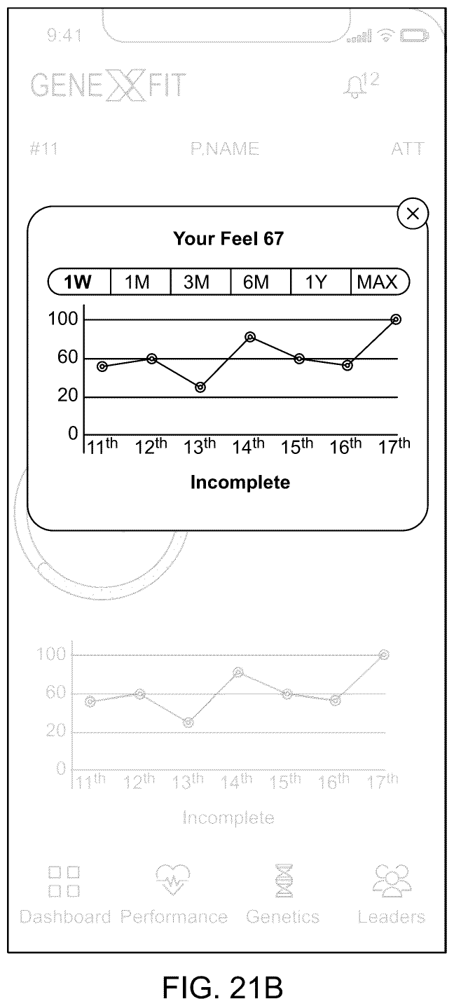

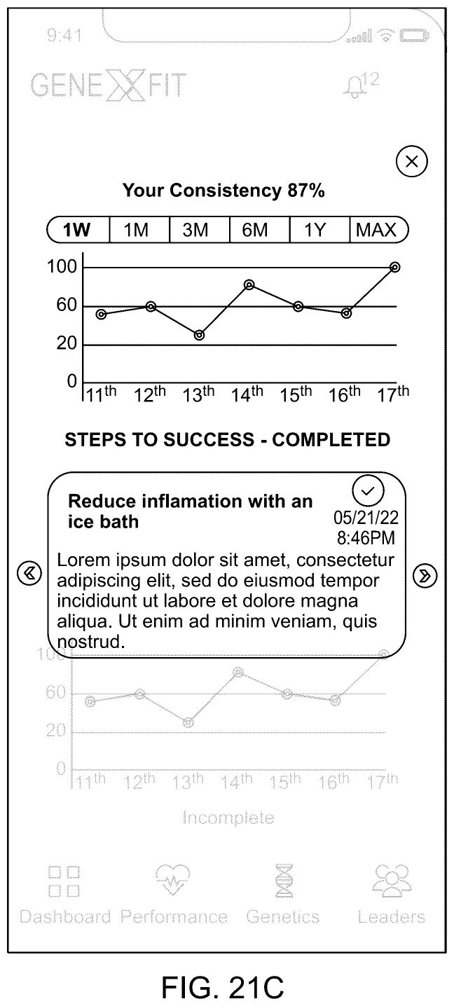

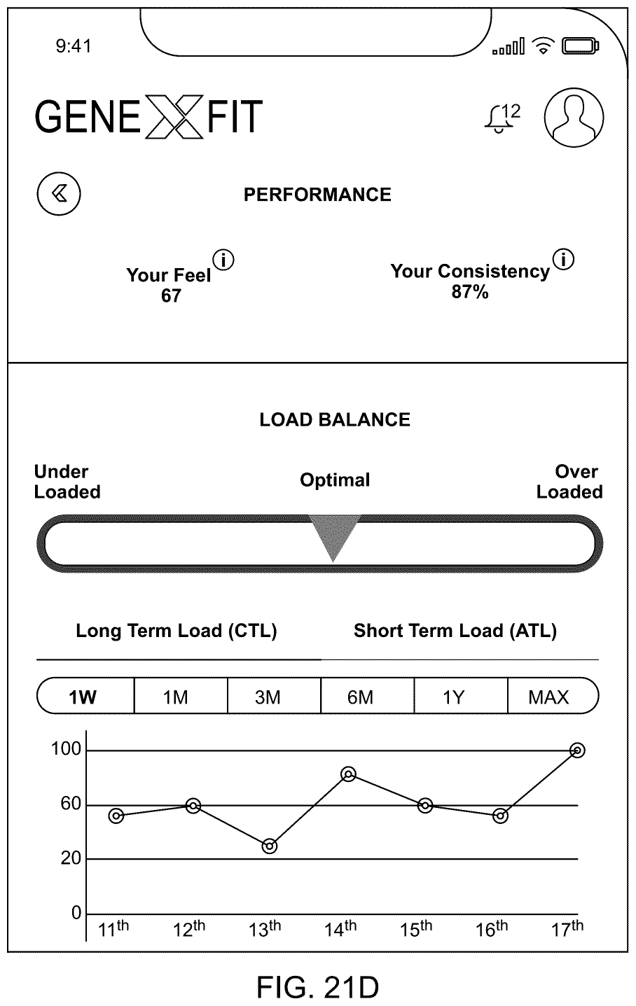

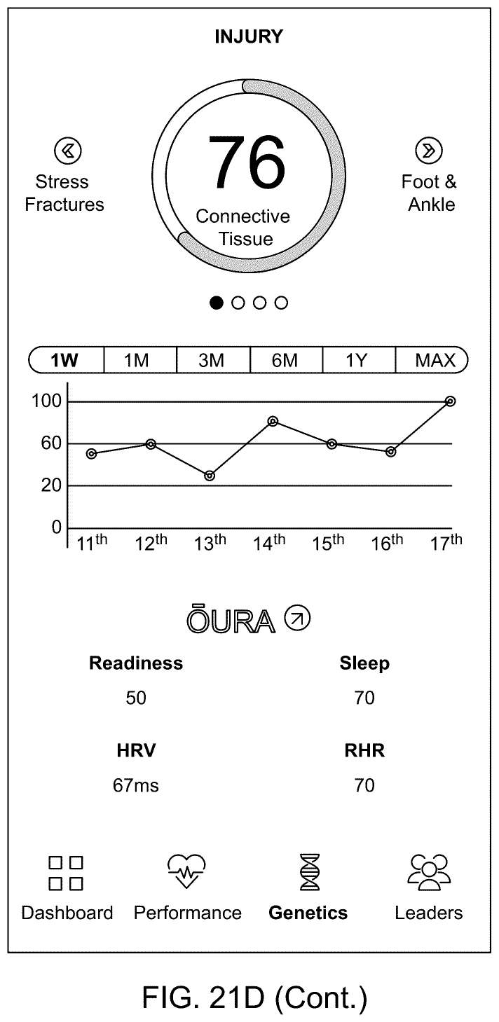

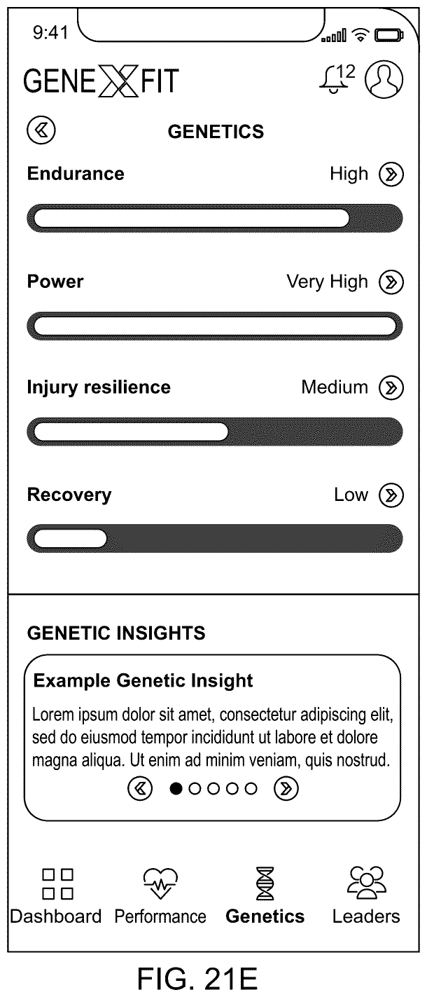

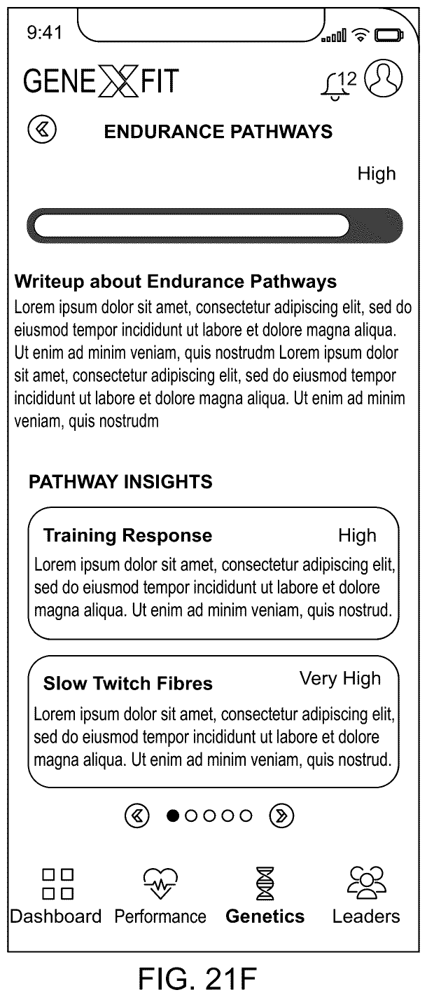

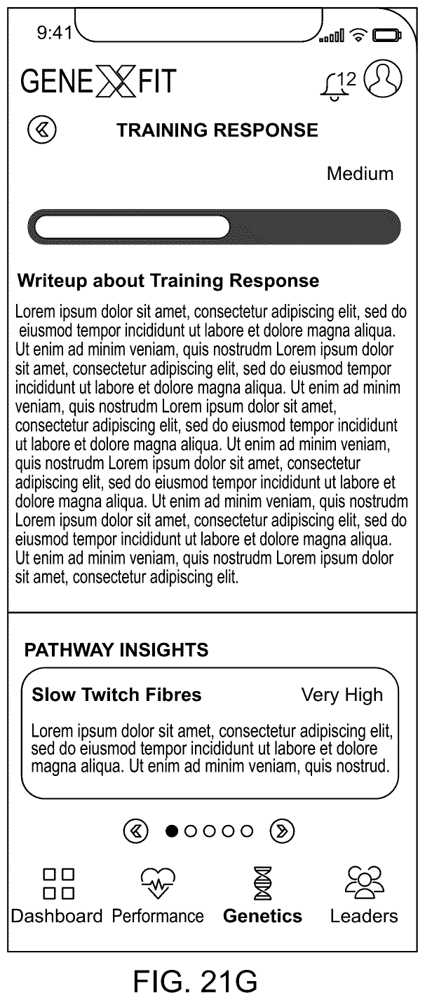

FIGS. 21A-21G show examples of various dashboard views of a software application user interface from a user perspective of a player (e.g., subject).

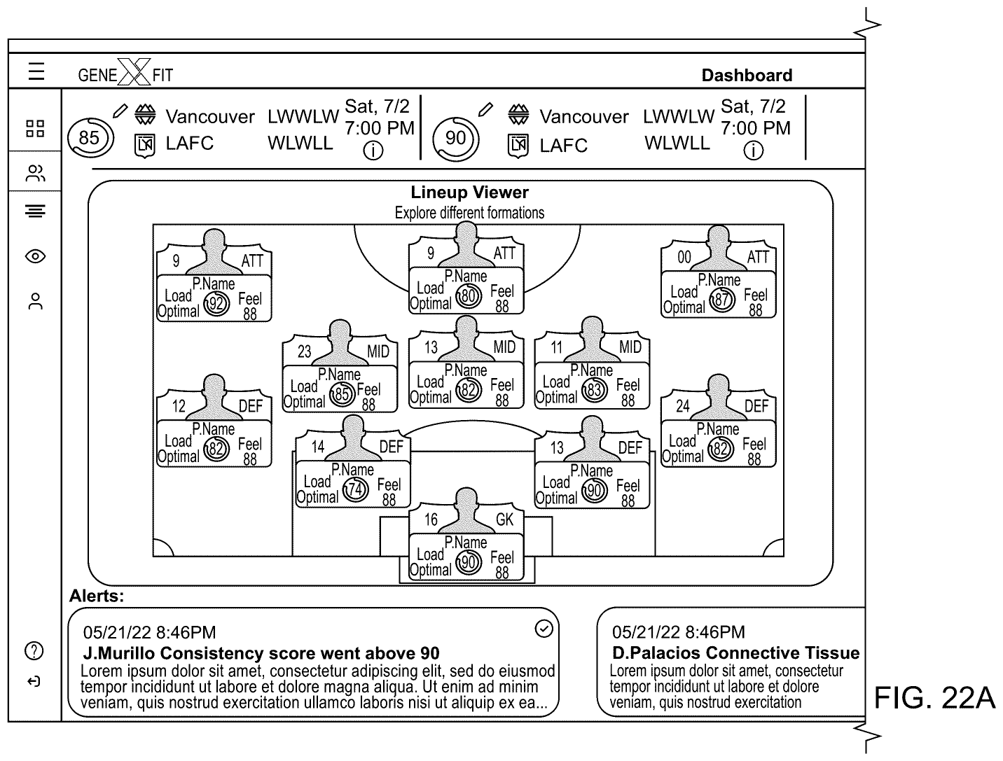

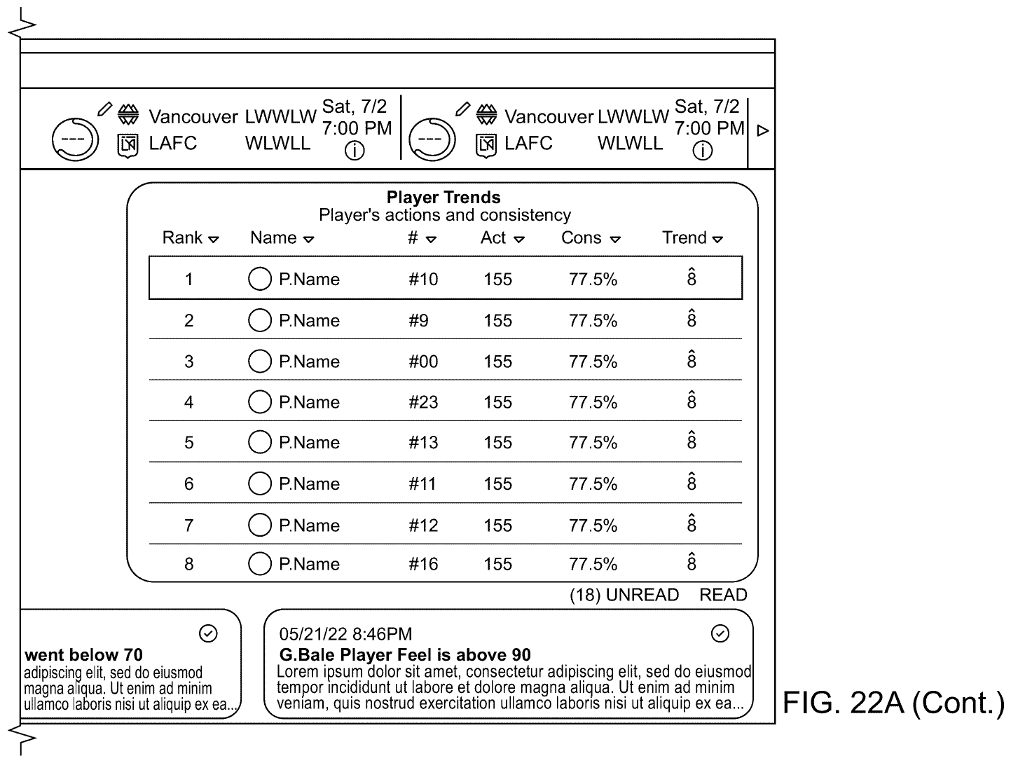

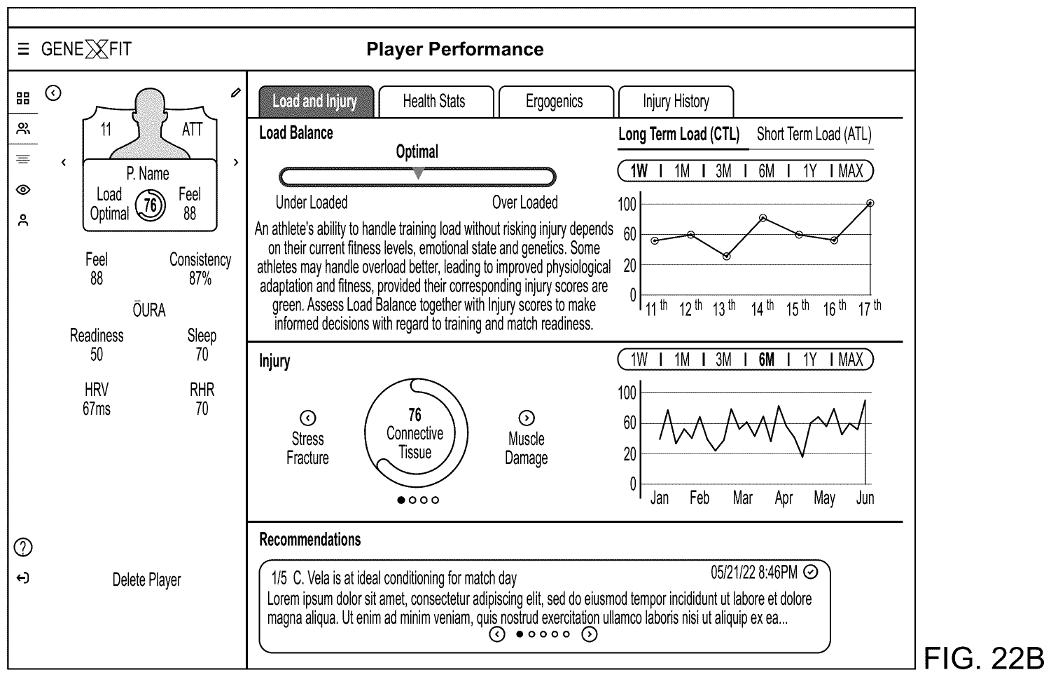

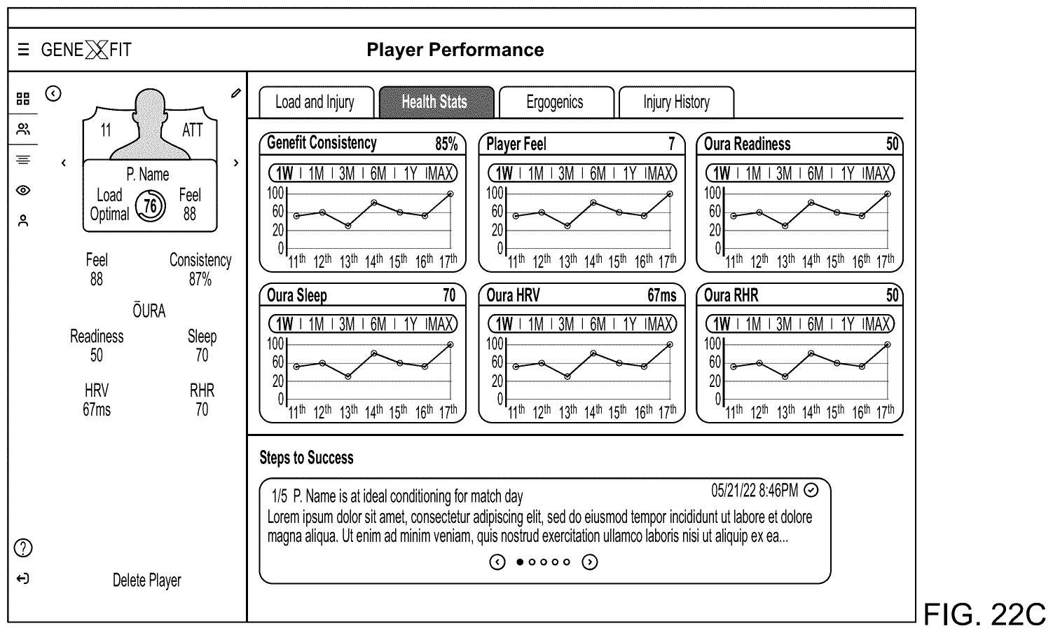

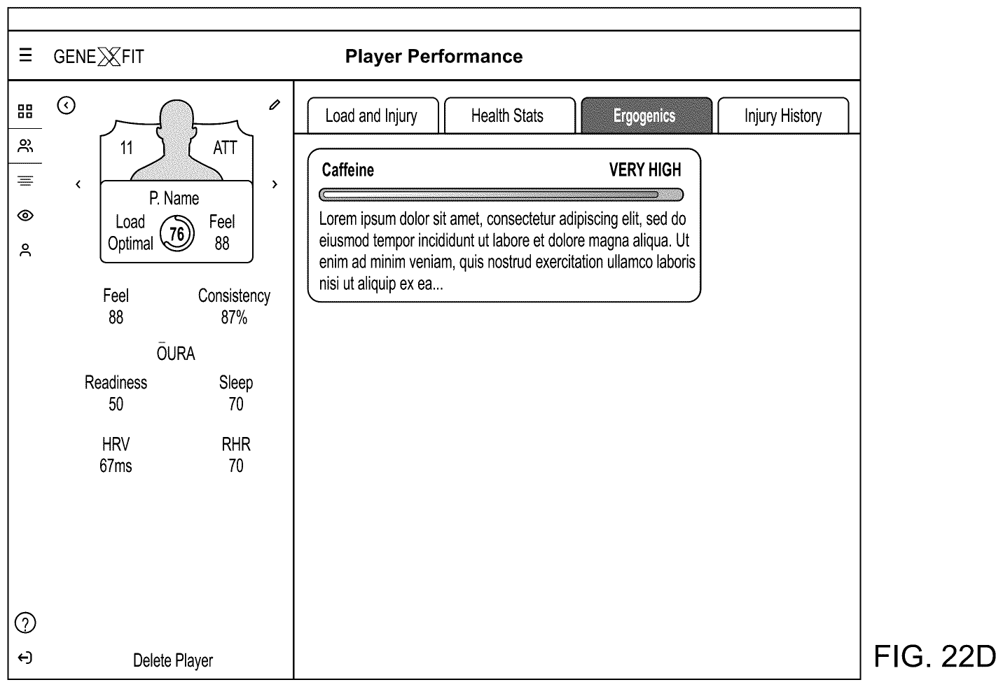

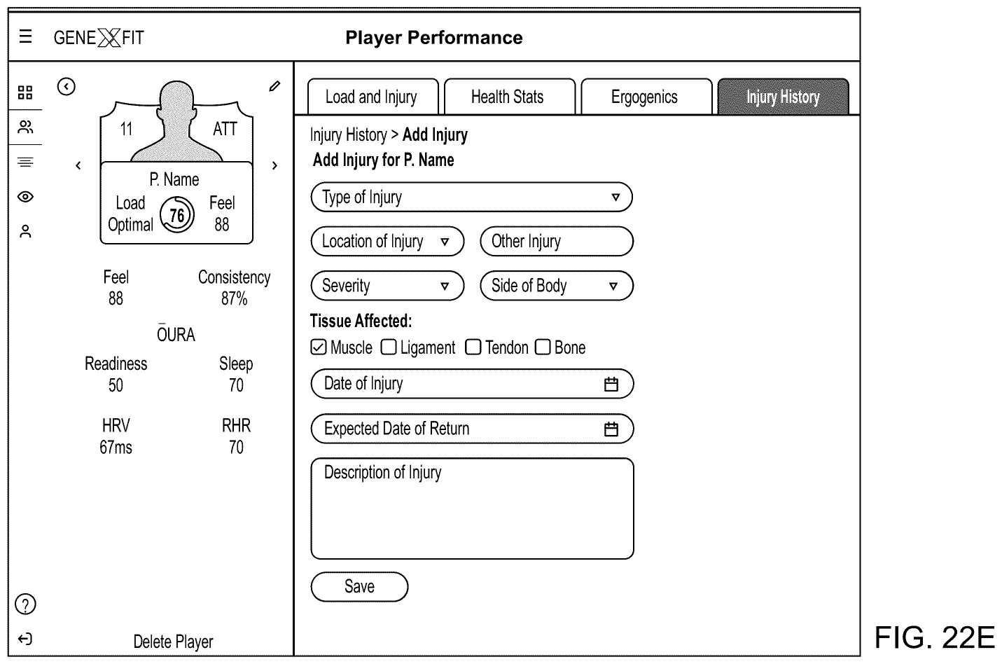

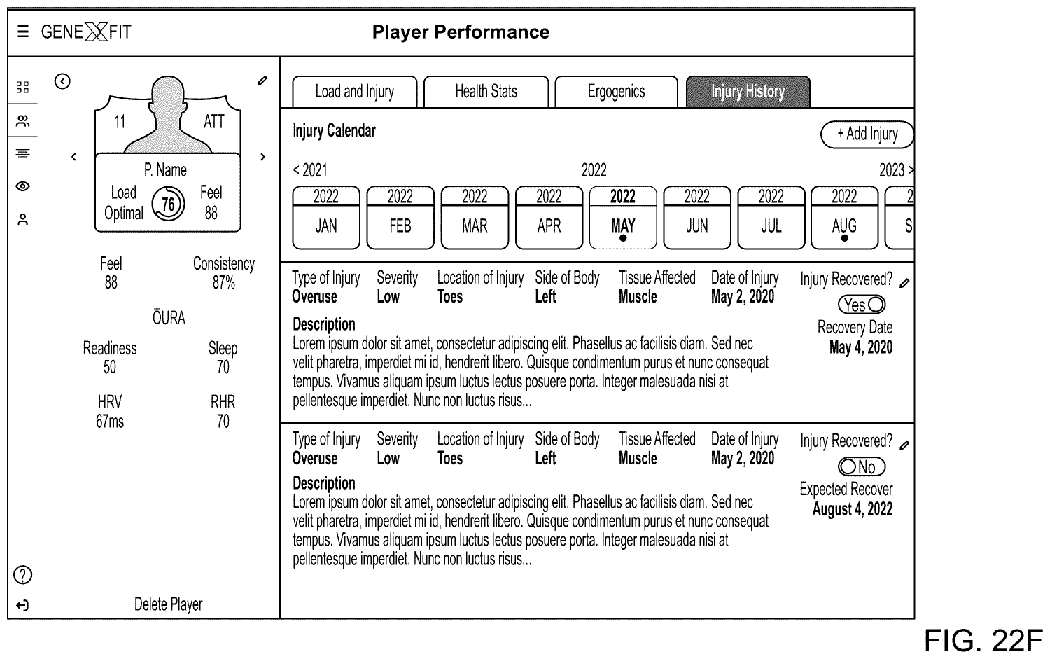

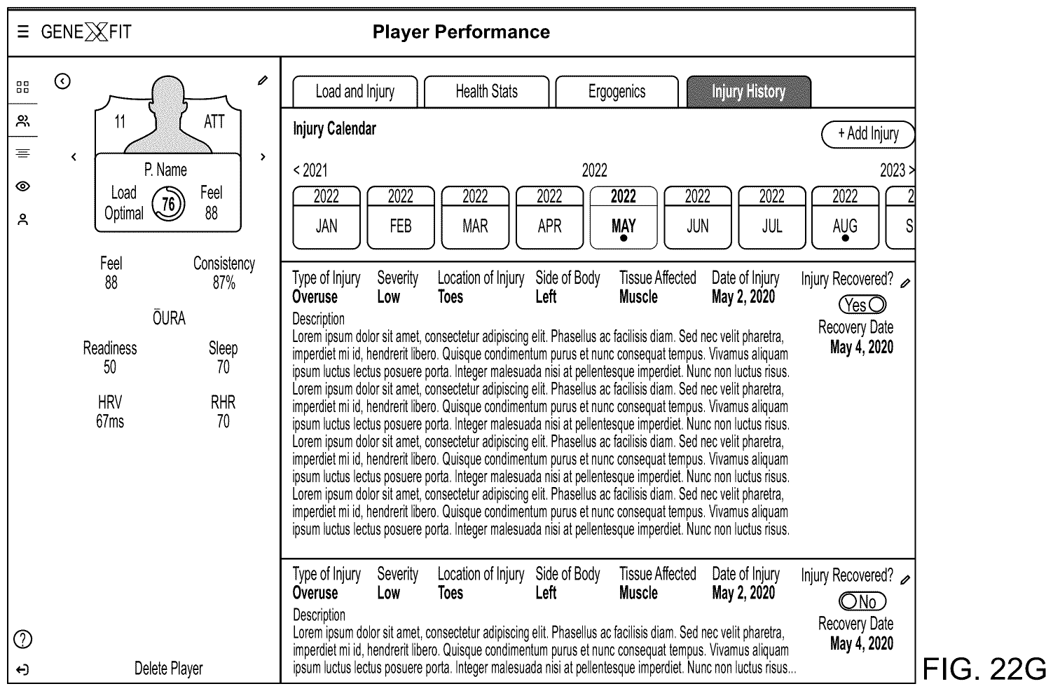

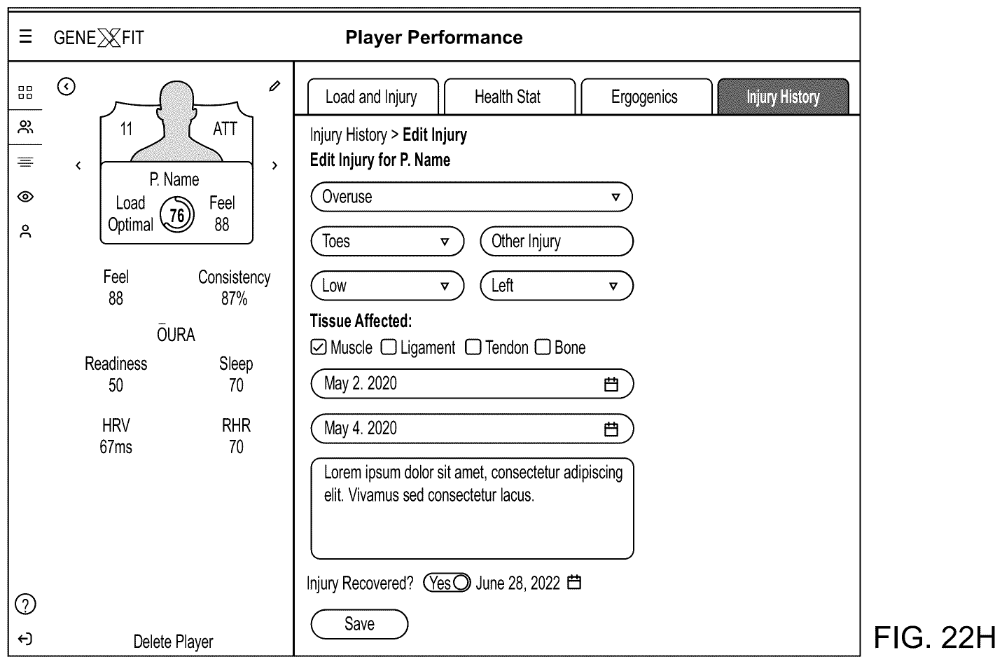

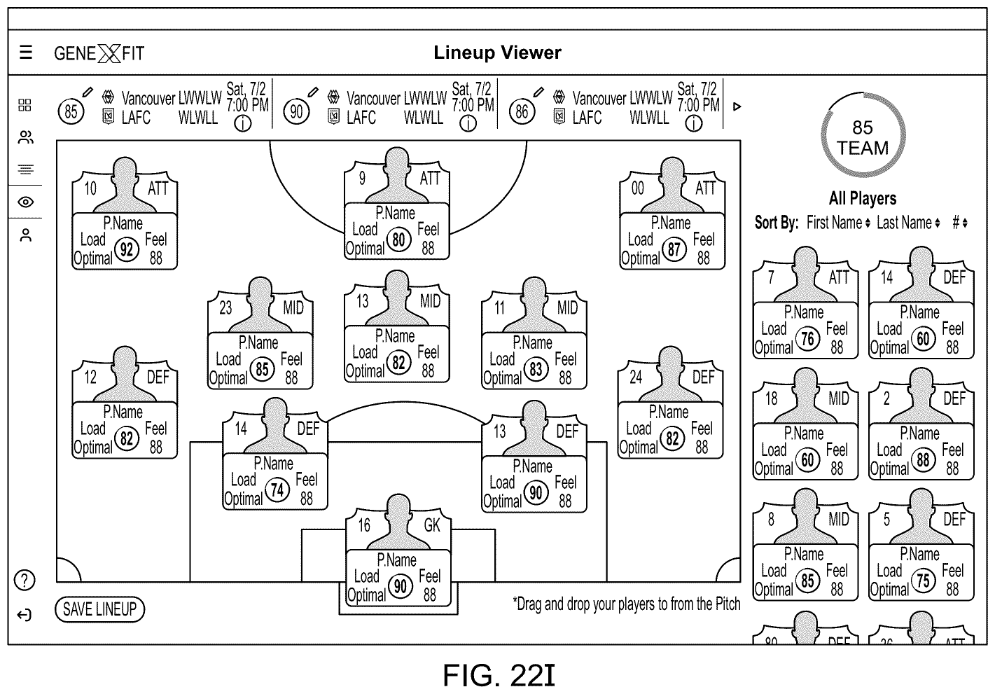

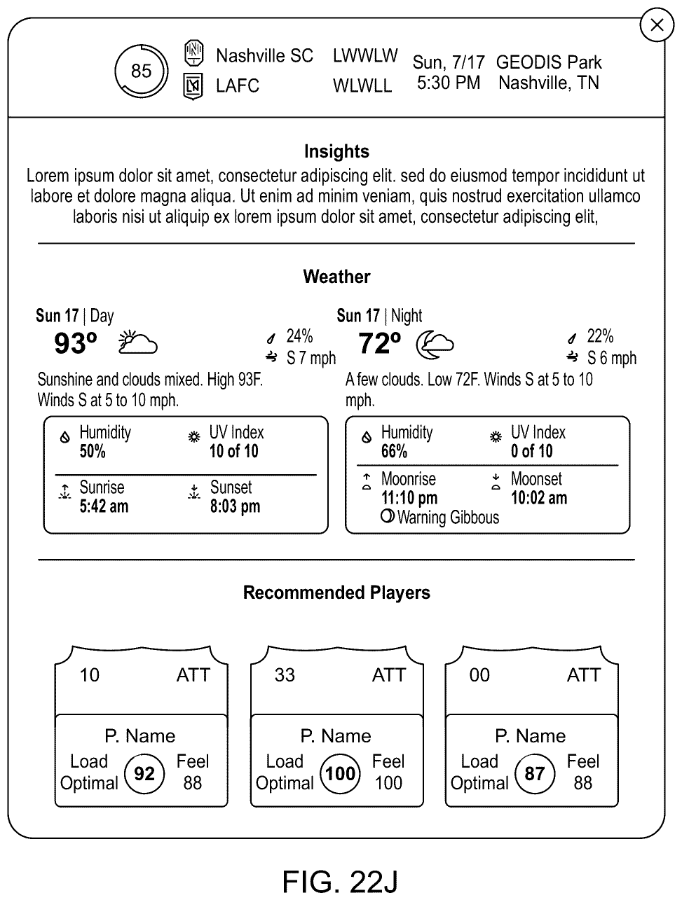

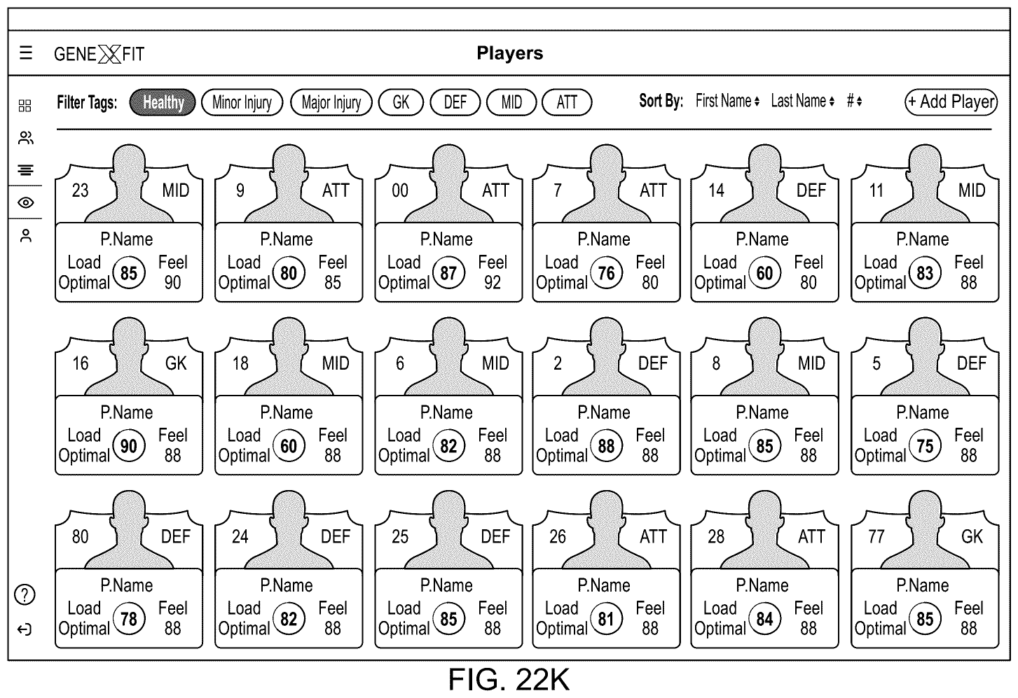

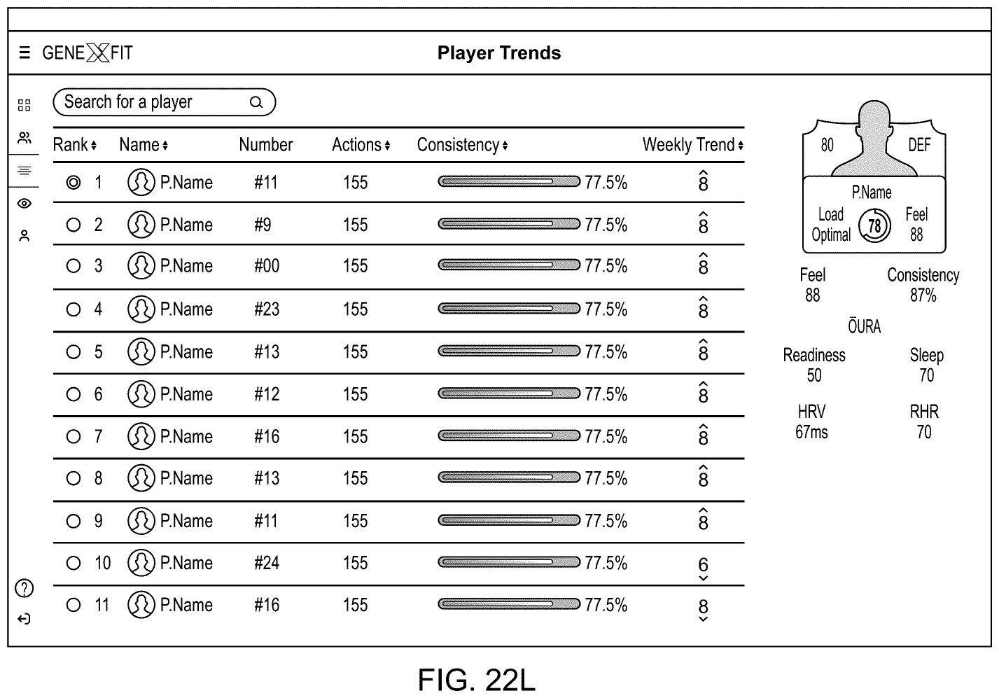

FIGS. 22A-22L show examples of various views of a software application user interface from a user perspective of a manager.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-55. (canceled)

56. A computer-implemented method for determining a performance or health risk state of a subject, comprising:
(a) receiving genetic information of said subject, wherein said genetic information is obtained by assaying a biological sample obtained or derived from said subject;
(b) receiving environmental information of said subject, wherein said environmental information comprises contextual data, activities, or physiological measurements of said subject;
(c) processing said genetic information and said environmental information to determine a performance or health risk state of said subject; and
(d) outputting an electronic report indicative of said performance or health risk state of said subject.

57. The method of claim 56, wherein said performance or health risk state comprises a performance or health risk score, number, or quantitative metric of said subject.

58. The method of claim 56, wherein said genetic information comprises nucleic acid sequence data, and wherein said nucleic acid sequence data comprises deoxyribonucleic acid (DNA) sequence data, ribonucleic acid (RNA) sequence data, or a combination thereof.

59. The method of claim 56, wherein said genetic information comprises genetic variants of said subject, and wherein said genetic variants comprise at least one of: single nucleotide polymorphisms (SNPs), copy number variants (CNVs), insertions or deletions (indels), fusions, and translocations.

60. The method of claim 56, wherein said biological sample is selected from the group consisting of saliva, cheek swab, blood, plasma, serum, urine, and a combination thereof.

61. The method of claim 56, wherein said assaying comprises at least one of: a single nucleotide polymorphism (SNP) panel assay, a pharmacology genetics assay, an ancestry genetics assay, a medical genetics assay, a pharmacogenetics assay, a sports performance genetics assay, a health screening, a test for a specific disease risk, a migraine test, a thyroid test, an eczema test, and a cancer genetics assay.

62. The method of claim 56, wherein said activities comprise at least one of: exercising, playing a sport, walking, running, sitting, standing, lying down, and sleeping.

63. The method of claim 56, wherein said physiological measurements comprise vital sign measurements of said subject, and wherein said vital sign measurements comprise at least one of: heart rate, heart rate variability, systolic blood pressure, diastolic blood pressure, respiratory rate, blood oxygen concentration (SpO2), carbon dioxide concentration in respiratory gases, a hormone level, sweat analysis, blood glucose, body temperature, impedance, conductivity, capacitance, resistivity, electromyography, galvanic skin response, neurological signals, and immunology markers.

64. The method of claim 56, wherein said physiological measurements comprise sports performance measurements of said subject, and wherein said sports performance measurements comprise at least one of: VO2max, blood lactate, lactate threshold, training load, training stress scores, times spent in aerobic and anaerobic heart rate zones, pace, power, distance, and time.

65. The method of claim 56, wherein said physiological measurements comprise a physiological metric that measures an effect of an external influence on a human body.

66. The method of claim 56, wherein said activities or said physiological measurements are obtained using an electronic device, and wherein said electronic device comprises a wearable device.

67. The method of claim 56, wherein (c) further comprises determining said performance or health risk state of said subject based at least in part on one or more of: a diagnosis of a disease or disorder, a prognosis of a disease or disorder, a risk of having a disease or disorder, a risk of sports-related injury, a treatment history of a disease or disorder, a history of previous treatment for a disease or disorder, a history of prescribed medications, a history of prescribed medical devices, age, height, weight, sex, smoking status, injury risk, training load status, fitness level, race or match readiness, and one or more symptoms of said subject.

68. The method of claim 56, further comprising generating a health regimen or a training regimen for said subject based at least in part on said performance or health risk state determined in (c).

69. The method of claim 68, wherein said health regimen comprises (i) recommendations to prevent onset of a disease or disorder, delay onset of a disease or disorder, reverse a disease or disorder, prevent injury, or maintain a physiological or health state of said subject, (ii) recommendations related to one or more of: diet, exercise, sports training, supplements, functional tests, blood tests, brain management, behavior change, skin care, environmental exposure, stress management, and mental health, or (iii) a combination thereof.

70. The method of claim 56, wherein said electronic report is presented on a graphical user interface of an electronic device of a user, wherein said graphical user interface is presented via a software application, and wherein said software application comprises a mobile software application.

71. The method of claim 56, further comprising transmitting said electronic report to a remote user, wherein said remote user is a clinical practitioner, a nutrigenetics counselor, a sports coach, a team manager, or an individual.

72. The method of claim 56, wherein (c) comprises processing said genetic information and said environmental information using a trained algorithm to determine said performance or health risk state of said subject.

73. The method of claim 72, wherein said trained algorithm comprises a supervised machine learning algorithm, and wherein said supervised machine learning algorithm comprises a deep learning algorithm, a support vector machine (SVM), a neural network, a Gaussian naïve Bayes model, a naïve Bayes model, or a Random Forest.

74. The method of claim 72, wherein said trained algorithm comprises an unsupervised machine learning algorithm, and wherein said unsupervised machine learning algorithm comprises a k-means clustering model or principal component analysis.

75. The method of claim 56, wherein said electronic report comprises a graphical representation of said performance or health risk state of said subject, and wherein said graphical representation comprises a time-series graph illustrating performance or health risk scores of a subject over time.

76. The method of claim 56, further comprising using said electronic report to provide said subject with a therapeutic intervention, wherein said therapeutic intervention comprises a drug.

77. The method of claim 56, wherein said performance or health risk state of said subject comprises a risk score, and wherein said method further comprises (i) using said risk score to modify a physiological estimation or measurement of said subject, and (ii) determining said performance or health risk state based at least in part on said modified physiological estimation or measurement of said subject.

78. The method of claim 77, further comprising generating a health regimen or a training regimen for said subject to decrease risk or improve performance, based at least in part on said modified physiological estimation or measurement of said subject.

79. The method of claim 78, wherein said health regimen comprises (i) recommendations to prevent onset of a disease or disorder, delay onset of a disease or disorder, reverse a disease or disorder, prevent injury, or maintain a physiological or health state of said subject, (ii) recommendations related to one or more of: diet, exercise, sports training, supplements, functional tests, blood tests, brain management, behavior change, skin care, environmental exposure, stress management, and mental health, or (iii) a combination thereof.

80. The method of claim 56, further comprising generating an updated performance or health risk state responsive to said subject following said health regimen.

81. The method of claim 56, wherein said electronic report further comprises subject-specific health and fitness recommendations, and wherein said subject-specific health and fitness recommendations comprise recommendations to improve a score or decrease a risk of said subject.

82. A system for determining a performance or health risk state of a subject, comprising:
a database configured to store genetic information of said subject, wherein said genetic information is obtained by assaying a biological sample obtained or derived from said subject, and environmental information of said subject, wherein said environmental information comprises contextual data, activities, or physiological measurements of said subject; and
one or more computer processors operatively coupled to said database, wherein said one or more computer processors are individually or collectively programmed to:
(i) process said genetic information and said environmental information to determine a performance or health risk state of said subject; and
(ii) electronically output a report indicative of said performance or health risk state of said subject.

83. A non-transitory computer-readable medium comprising machine-executable code that, upon execution by one or more computer processors, implements a method for determining a performance or health risk state of a subject, said method comprising:
(a) receiving genetic information of said subject, wherein said genetic information is obtained by assaying a biological sample obtained or derived from said subject;
(b) receiving environmental information of said subject, wherein said environmental information comprises contextual data, activities, or physiological measurements of said subject;
(c) processing said genetic information and said environmental information to determine a performance or health risk state of said subject; and
(d) outputting an electronic report indicative of said performance or health risk state of said subject.
